(19) United States
(12) Patent Application Publication
(10) Pub. No.: US 2022/0265574 A1
(43) Pub. Date: Aug. 25, 2022

(54) METHODS OF USING CANNABIDIOL AND A STEROID

(71) Applicant: STERO BIOTECHS LTD., RAMAT GAN (IL)

(72) Inventors: Moshe YESHURUN, Elqana (IL); Sari Prutchi SAGIV, Kibbutz HaMaapil (IL)

(21) Appl. No.: 17/744,996

(22) Filed: May 16, 2022

Related U.S. Application Data
(63) Continuation-in-part of application No. 16/048,856, filed on Jul. 30, 2018, now Pat. No. 11,357,740.
Application No. 16/048,856 is a continuation of application No. 15/877,665, filed on Jan. 23, 2018, now Pat. No. 10,058,515.
Application No. 15/877,665 is a continuation of application No. 15/143,694, filed on May 2, 2016, now Pat. No. 9,889,100.
Application No. 15/143,694 is a continuation-in-part of application No. 14/787,515, filed on Oct. 28, 2015, now Pat. No. 9,956,182.
(60) Provisional application No. 61/818,525, filed on May 2, 2013.

(51) Int. Cl. A61K 31/05; A61K 9/00; A61K 45/06

(52) U.S. Cl. CPC A61K 31/05; A61K 9/0053; A61K 9/0014; A61K 45/06; A61K 9/0095

(57) ABSTRACT

The invention provides methods for preventing, ameliorating and treating inflammatory diseases with a reduced dose of a steroid by providing a combined cannabidiol (CBD) and steroid therapy.

### CROSS-REFERENCE TO RELATED APPLICATIONS

This is a continuation-in-part of U.S. patent application Ser. No. 16/048,856 filed Jul. 30, 2018, which is a continuation of U.S. patent application Ser. No. 15/877,665, filed on Jan. 23, 2018, now U.S. Pat. No. 10,058,515, which is a continuation of U.S. patent application Ser. No. 15/143,694, filed on May 2, 2016, now U.S. Pat. No. 9,889,100, which is a continuation-in-part of U.S. patent application Ser. No. 14/787,515, filed on Oct. 28, 2015, now U.S. Pat. No. 9,956,182, which is a national phase of PCT Patent Application No. PCT/IL2014/050385, filed on Apr. 29, 2014, which claims the benefit of priority of U.S. Provisional Patent Application No. 61/818,525, filed on May 2, 2013. The foregoing patent applications are incorporated by reference in their entirety herein.

### FIELD

The present invention relates to methods and uses of Cannabidiol compositions with steroid therapy.

### BACKGROUND

Cortisone was first discovered in the 1920s through animal research at the Mayo Clinic (1). A decade later, a group of endocrinologists at the Mayo Clinic, led by Dr. Edward C. Kendall, began investigating the chemical and physiologic components of the adrenal cortex to better understand this hormone. In 1934, the first compounds were separated and given the names Compounds A to D. Subsequently, in 1935 the other 2 compounds were isolated and named Compounds E and F.

The anti-inflammatory effect of corticosteroids correlates with dose and duration of treatment. The mechanism is multifactorial but is ultimately most dependent on 2 mechanisms of action: (1) the inhibition of cytokine, chemokine, and adhesion molecule production and (2) the antagonism of the action of proinflammatory cytokines, including interleukin-1 and tumor necrosis factor. These actions are achieved through inhibition of transcription factors AP-1 and NFkB through several mechanisms that vary based on cell type. In some cells, inhibition occurs through direct protein interactions, resulting in decreased transcriptional activity; for instance, some corticosteroids inhibit Janus kinase signal transduction and activation transcription pathways that are usually initiated by IL-2 and interferon-gamma.

In other cells, inhibition occurs through induction of inhibitory molecules such as lipocortin-1 (a phospholipase A2 inhibitor which blocks eicosanoid generation and cyclooxygenase-2 induction). Through these mechanisms, corticosteroids inhibit vasodilation and vascular permeability, resulting in decreased plasma exudation, erythema, and swelling

The administration of corticosteroids also affects white cell response. For example, corticosteroids cause neutrophilic leukocytosis. Though the exact mechanism is not completely understood, it is hypothesized that corticosteroids alter adhesion molecules of neutrophils or the endothelial surface (E-selectin and ICAM-1), which prevents neutrophils from reaching the inflammatory site. Additionally, there is a decrease in circulating levels of eosinophils, basophils, monocytes, and lymphocytes (decrease in T more than B and CD4 more than CD8) in response to corticosteroids.

It is not completely understood, but research has shown that there is down regulation of lymphocyte adhesion molecules (such as LFA-1 and CD2) through action at corticosteroid receptors. There is also evidence of nongenomic effects of corticosteroids related to cell membrane redistribution and changes to the cellular adhesion molecules themselves, as well as redistribution of lymphocytes to bone marrow, spleen, skin, and regional lymph nodes (Stone S. et al. (2021) Pain Physician 2021; 24:S233-S246).

Hence, corticosteroids are first-line medications for treating inflammatory conditions and diseases. Steroids affect the immune system as well as other organs and systems.

The majority of the side-effects of steroids are unavoidable and predictable. In other words, a given steroid treatment for controlling a disease such as a chronic disease requires a steroid dose and steroid duration of treatment which likely induce devastating side effects.

Despite the devastating side-effects of corticosteroids, therapy involving a corticosteroid is considered an essential irreplaceable therapy for many diseases, including Crohn's Disease. It can be said that corticosteroids are lifesaving drugs that often negatively affect many organs and system and can even cause a disease.

### SUMMARY

According to one aspect the present invention there is provided a method for enhancing the therapeutic effect of a steroid or reducing the effective dose of the steroid in a subject afflicted with acute respiratory distress syndrome (ARDS), inflammatory bowel disease (IBD), or Urticaria, comprising administering to the subject the steroid and cannabidiol (CBD), thereby enhancing the therapeutic effect of the steroid in the subject afflicted with acute respiratory distress syndrome (ARDS), inflammatory bowel disease (IBD), or Urticaria.

According to some embodiments of the invention, enhancing the therapeutic effect of the steroid comprises: reducing a side effect associated with the steroid, reducing the effective dose of the steroid or rendering a refractory steroid dose a therapeutic effective dose.

According to some embodiments of the invention, steroid, CBD, or any combination thereof is administered systemically. In some embodiments, the steroid, CBD, or any combination thereof is administered orally or topically. In some embodiments, CBD is administered daily in doses of between 5 mg to 600 mg.

According to some embodiments of the invention, enhancing the therapeutic effect of the steroid is enhancing the recovery of a subject afflicted with ARDS or reducing death risk of the subject afflicted with ARDS. In some embodiments, ARDS is COVID-19. In some embodiments, the subject afflicted with ARDS is treated with respiratory support, ventilatory support or a combination thereof.

According to one aspect the present invention there is provided a method of tapering off steroid therapy in a subject afflicted with a chronic inflammatory disease, comprising the following consecutive steps: (a) administering to the subject CBD and effective dose of said steroid; (b) gradually decreasing the dose of the steroid and maintaining co-administration of CBD, wherein the rate of said decreasing is matched to controlling the chronic inflammatory disease; and (c) tapering off the steroid therapy, once the chronic inflammatory disease is controlled, inhibited, treated or ameliorated, while administering CBD. In some embodiments, chronic inflammatory disease is an inflammatory bowel disease (IBD) or Urticaria. In some embodiments, IBD is Crohn's disease. In some embodiments, inhibited comprises inhibiting flares. In some embodiments, tapering off the steroid therapy is ceasing the steroid therapy.

According to one aspect the present invention the chronic inflammatory disease in the subject destined to tapering off steroid therapy, is controlled, inhibited, treated or ameliorated by the effective dose of the steroid. In some embodiments, CBD dose in step (b) is increased concomitantly with decreasing the dose of the steroid.

According to yet another aspect there is provided a method for enhancing the therapeutic effect of a steroid in a subject in need of a steroid therapy, comprising administering to the subject the steroid and a cannabidiol (CBD) or a functional derivative thereof.

### DETAILED DESCRIPTION

Described herein is the surprising observation that administration of a composition comprising Cannabidiol or a functional derivative thereof to transplanted patients experiencing the symptoms of, and diagnosed with severe forms of acute and chronic graft-versus-host-disease (GVHD), showed significant improvement in the disease severity, halted disease progression and even complete remission of its symptoms. Accordingly, provided herein are methods for the treatment GVHD and its symptoms, in its severest forms, by administration of CBD to a subject in need thereof.

### I. Graft Versus Host Disease

Graft-versus-host-disease (GVHD) is a life-threatening, complication that can arise following allogeneic hematopoietic cell transplantation. GVHD is the leading cause of post-transplantation morbidity and non-relapse mortality in hematopoietic stem cell transplants (HSCTs) and poses the greatest threat to transplantation success (Pasquini and Wang, 2013).

There are two main categories of GVHD, each with two subcategories as outlined in Table 1, below. The broad category of acute GVHD includes (1) classic acute GVHD (maculopapular rash, nausea, vomiting, anorexia, profuse diarrhea, ileus, or cholestatic hepatitis) occurring within 100 days after transplantation or Donor Lymphocyte Infusion (DLI) without diagnostic or distinctive signs of chronic GVHD, and (2) persistent, recurrent, or late-onset acute GVHD (features of classic acute GVHD without diagnostic or distinctive manifestations of chronic GVHD) occurring beyond 100 days of transplantation or DLI (often seen after withdrawal of immune suppression).

The broad category of chronic GVHD includes (1) classic chronic GVHD without features characteristic of acute GVHD and (2) an overlap syndrome in which features of chronic and acute GVHD appear together. In the absence of histologic or clinical signs or symptoms of chronic GVHD, the persistence, recurrence, or new onset of characteristic skin, GI tract, or liver abnormalities should be classified as acute GVHD regardless of the time after transplantation. With appropriate stratification, patients with persistent, recurrent, or late-onset acute GVHD or overlap syndrome can be included in clinical trials with patients who have chronic GVHD (Filipovich et al, 2005).

TABLE 1
Categories of Acute and Chronic GVHD

| | | | |
|---|---|---|---|
| | Time of | Presence of | Presence of |
| | symptoms after | aGVHD | cGVHD |
| Category | HCT or DLI | features | features |

Acute GVHD

| | | | |
|---|---|---|---|
| Classic aGVHD | ≤100 d | Yes | No |
| Persistent, | >100 d | Yes | No |
| recurrent or late- | | | |
| onset aGVHD | | | |

Chronic GVHD

| | | | |
|---|---|---|---|
| Classic cGVHD | No time limit | No | Yes |
| Overlap | No time limit | Yes | Yes |
| syndrome | | | |

Adapted from Filipovich et al, 2005

### Acute GVHD

Clinically, acute GVHD, treated by the methods described herein, is suspected when a recipient of HSCT develops any or all of the following signs or symptoms: dermatitis (skin rash), cutaneous blisters, crampy abdominal pain with or without diarrhea, persistent nausea and vomiting, or hepatitis (with elevation of bilirubin and/or liver enzymes). Typically, these symptoms start with donor engraftment and may occur up to day 100 after the HSCT, but may also occur later. Acute GVHD is a clinical diagnosis but, as many the symptoms of acute GVHD are non-specific, histologic confirmation, especially if the symptoms are atypical or involve just the liver or gut, may be extremely useful (Jacobsohn & Voselsang, 2007).

As shown in Table 2, the clinical manifestations (also described herein as the “associated conditions” or “associated symptoms”) of acute GVHD occur in the skin, gastrointestinal tract and liver (Vogelsang et al, 2003). In a comprehensive review, Martin et al found that at the onset of acute GVHD, 81% of patients had skin involvement, 54% had GI involvement, and 50% had liver involvement (Martin et al, 1990).

### Table 2: Acute GVHD Symptoms

TABLE 2
Acute GVHD symptoms

| | |
|---|---|
| Skin | Maculopapular skin rash |
| Upper GI tract | Nausea and/or anorexia + positive histology |
| Lower GI tract | Watery diarrhea ≥500 mL ± severe abdominal pain ± |
| | bloody diarrhea or ileus (after exclusion of infectious |
| | etiology) |
| Liver | Cholestatic hyperbilirubinemia |

Adapted from Ferrara et al, 2009

The incidence of the severity of acute GVHD is determined by the extent of involvement of the three principal target organs (see Table 2 above). The overall characteristics of the stages and grades of acute GVHD (aGVHD) are listed in Table 3 (Jacobsohn & Vogelsang, 2007). Severe acute GVHD carries a poor prognosis, with 25% long term survival for grade III and 5% for grade IV (Cahn et al, 2005).

TABLE 3
Skin, liver and GI characteristics
of the stages and grades of aGVHD

| | | | | Gut (stool |
|---|---|---|---|---|
| Stage | Skin | Liver (bilirubin) | | output/day) |
| 0 | No GVHD rash | <2 | mg % | <500 mL/day or |
| | | | | persistent nausea |
| 1 | Maculopapular | 2-3 | mg % | 500-999 |
| | rash | | | mL/day |
| | <25% BSA | | | |
| 2 | Maculopapular | 3.1-6 | mg % | 1000-1500 |
| | rash | | | mL/day |
| | 25-50% BSA | | | |
| 3 | Maculopapular | 6.1-15 | mg % | Adult: >1500 |
| | rash | | | mL/day |
| | >50% BSA | | | |
| 4 | Generalized | >15 | mg % | Severe abdominal |
| | erythroderma + | | | pain with or |
| | bullous formation | | | without ileus |

Grade

| | | | |
|---|---|---|---|
| I | Stage 1-2 | None | None |
| II | Stage 3 or (go to | Stage 1 or (go to | Stage 1 |
| | next column) | next column) | |
| III | — | Stage 2-3 or (go | Stage 2-3 |
| | | to next column) | |
| IV | Stage 4 or (go to | Stage 4 | — |
| | next column) | | |

Adapted from Jacobsohn & Voselsang, 2007

### Treatments for Acute GVHD

Corticosteroids remain the standard first-line treatment for patients who fail prevention and develop acute GVHD. The majority of centers use methylprednisolone (MP) at doses of 1-2 mg/kg body weight. The present invention, in some embodiment, combines treatment with a steroid such as MP with Cannabidiol or a functional derivative thereof. The response to primary treatment is of central importance since responses correlate with survival. Patients not responding to steroids have a dismal prognosis. It will be appreciated that clinical definitions change over time, however, the current definition of nonresponse or refractoriness to first line aGVHD therapy is: progression after 3 days, no improvement after 7 days, or incomplete response after 14 days. Patients refractory to first line therapy are offered second-line treatment. However, there is no standard second-line treatment for acute GVHD. Widely used components are MMF, anti-TNF antibodies, other monoclonal antibodies, ATG, extracorporeal photopheresis, MTX and mesenchymal stem cells. Despite partial or complete response rates of 35%-70% with second-line medications, survival at 6-12 months is dismal, only 30%. As described further herein, the current disclosure relates to administration of CBD and derivatives thereof to subjects that have aGVHD, including severe aGVHD. Such patients include those classified as having Grade III or Grade IV GVHD, and/or patients who are refractory to other treatments. In one embodiment, refractory GVHD is steroid refractory GVHD—the steroid treatment does not treat, ameliorate or improve a clinical outcome or a clinical score in a subject afflicted with GVHD. In one embodiment, steroid refractory GVHD is characterized by deterioration in at least one GVHD symptom or a condition associated with GVHD as further described herein. In one embodiment, steroid refractory GVHD is characterized by deterioration in at least one GVHD symptom or a condition associated with GVHD after 2 days of a steroid treatment. In one embodiment, steroid refractory GVHD is characterized by deterioration in at least one GVHD symptom or a condition associated with GVHD after 3 days of a steroid treatment. In one embodiment, steroid refractory GVHD is characterized by deterioration in at least one GVHD symptom or a condition associated with GVHD after 4 days of a steroid treatment. In one embodiment, steroid refractory GVHD is characterized by deterioration in at least one GVHD symptom or a condition associated with GVHD after 5 days of a steroid treatment. In one embodiment, steroid refractory GVHD is characterized by deterioration in at least one GVHD symptom or a condition associated with GVHD after 6 days of a steroid treatment.

In one embodiment, steroid refractory GVHD is characterized by lack of improvement in at least one GVHD symptom or a condition associated with GVHD after 4 to 10 days of a steroid treatment. In one embodiment, steroid refractory GVHD is characterized by lack of improvement in at least one GVHD symptom or a condition associated with GVHD after 5 to 9 days of a steroid treatment. In one embodiment, steroid refractory GVHD is characterized by lack of improvement in at least one GVHD symptom or a condition associated with GVHD after about (±2 days) 7 days of a steroid treatment.

In one embodiment, steroid refractory GVHD is characterized by lack of remission in GVHD and/or at least one GVHD symptom or a condition associated with GVHD after 8 to 21 days of a steroid treatment. In one embodiment, steroid refractory GVHD is characterized by lack of remission in GVHD and/or at least one GVHD symptom or a condition associated with GVHD after 10 to 18 days of a steroid treatment. In one embodiment, steroid refractory GVHD is characterized by lack of remission in GVHD and/or at least one GVHD symptom or a condition associated with GVHD after 12 to 16 days of a steroid treatment. In one embodiment, steroid refractory GVHD is characterized by lack of remission in GVHD and/or at least one GVHD symptom or a condition associated with GVHD after about (±2 days) 14 days of a steroid treatment.

### Chronic Graft-Versus-Host-Disease

Chronic GVHD is distinctive from acute GVHD and is not simply an evolution of acute GVHD. Chronic GVHD is a syndrome of variable clinical features resembling autoimmune and other immunologic disorders such as scleroderma. The pathophysiology of the chronic GVHD syndrome may involve inflammation, cell-mediated immunity, humoral immunity, and fibrosis. Clinical manifestations nearly always present during the first year after transplantation, but some cases develop many years after HCT. Manifestations of chronic GVHD may be restricted to a single organ or site or may be widespread, with profound impact on quality of life.

### Global Scoring of Chronic GVHD

Similar to the aGVHD “Grade” classification, Chronic GVHD severity score at baseline predicts overall survival and non-relapse mortality. The methods of the present invention, in some embodiment, enhances overall survival and/or reduces non-relapse mortality of a subject afflicted with GVHD.

Eight organs or sites (skin, mouth, eyes, gastrointestinal tract, liver, lungs, joint and fascia, and genital tract) are considered for calculating global score. The methods of the present invention, in some embodiment, treat the different global scores and the various organs involved in the GVHD. Elements included in the proposed global scoring include both the number of organs or sites involved and the severity score within each affected organ. The global descriptions of mild, moderate, and severe were chosen to reflect the degree of organ impact and functional impairment due to chronic GVHD. Although scoring is often used at the time of initial diagnosis, evaluating the clinical score periodically during the course of chronic GVHD may revise prognostic expectations and better describe the current severity of chronic GVHD. Mild chronic GVHD, treated by the current methods, is present when one or two organs show symptoms with no more than a score of 1 plus, and the lung score is 0. Moderate chronic GVHD, treated by the current methods, is present when three or more organs are involved with a score of 1 OR at least one organ is involved with a score of 2 or the lung score is 1. Lastly, severe chronic GVHD, treated by the current methods, is present when at least one organ has a score of 3 OR the lung score is 2 or 3.

Symptomatic mild chronic GVHD, treated by the current methods, may be managed with local therapies alone (e.g. topical corticosteroids for the skin involvement) and CBD or any functional derivative thereof. In patients with chronic GVHD that involves three or more organs or with a score of 2 or greater in any single organ, however, systemic immunosuppressive therapy is administered together with CBD or any functional derivative thereof. In some examples however, some organ sites (mouth, eyes, genital tract), aggressive local therapy alone may be reasonable together with CBD or any functional derivative thereof, as response to systemic therapy may be suboptimal or may not warrant the risk of treatment. Early intervention with effective systemic therapy together with CBD or any functional derivative thereof can treat or prevent progression to severe chronic GVHD or the treatment and/or escalation of severe chronic GVHD. Effective immune-modulating therapy together with CBD or any functional derivative thereof can ameliorate clinical manifestations and possibly prolong survival. In patients with newly diagnosed chronic GVHD already taking immune suppressive medications a CBD or any functional derivative thereof may further enhance the efficacy of treatment.

Chronic GVHD, treated by the present methods, is one of the major causes of late treatment related mortality (TRM) after allogeneic HCT. Prospective studies have shown that the skin score, lung score, and gastrointestinal score each predict the risk of TRM. Previous studies have identified several factors associated with an increased risk of TRM among patients with chronic GVHD, including involvement of multiple organs or sites, decreased clinical performance score, thrombocytopenia at the time of diagnosis, progressive onset of chronic GVHD from prior acute GVHD (or onset of chronic GVHD during steroid treatment), hyperbilirubinemia, a higher percentage of skin involvement at the time of diagnosis, and others. Characteristics consistently associated with an increased risk of late TRM among patients with chronic GVHD are thrombocytopenia and progressive onset of chronic GVHD from acute GVHD.

### Treatments for Chronic GVHD

Current treatments for chronic GVHD seek to relieve symptoms, control disease activity, and prevent damage and disability, but the efficacy of currently used local and/or systemic is mixed, with patients often refractory to treatment. In the current disclosure, it is shown that administration of cannabidiol to patients with severe refractory chronic GVHD experienced dramatic improvement, and in certain examples complete remission, of the GVHD as observable in treatment of the symptoms and related conditions.

### II. CBD Treatment of GVHD

According to one aspect, the present invention provides pharmaceutical compositions comprising CBD or any functional derivative thereof for use in methods of treating acute and/or chronic GVHD. In some embodiments, the invention provides a method for treating Grade III and/or Grade IV acute GVHD with a pharmaceutical composition of the invention. In some embodiments, the invention provides a method for treating severe chronic GVHD with a pharmaceutical composition of the invention.

The compositions specified herein below, comprise Cannabidiol (CBD), or any functional derivative thereof (i.e. a CBD derivative possessing similar, equivalent, or increased efficacy). In some embodiments, the described compositions optionally further comprise at least one pharmaceutically acceptable carrier, diluent, excipient and/or additive.

The phrase “CBD or any functional derivative thereof”, according to some embodiments, refers to compounds and/or compositions that are substantially and/or essentially devoid of THC. In one embodiment, a composition comprising CBD or any functional derivative thereof, as described herein is substantially and/or essentially devoid of THC.

The phrase “CBD or any functional derivative thereof”, according to some embodiments, refers to compounds and/or compositions that comprise at least 80% CBD or any functional derivative thereof. The phrase “CBD or any functional derivative thereof”, according to some embodiments, refers to compounds and/or compositions that comprise at least 90% CBD or any functional derivative thereof. The phrase “CBD or any functional derivative thereof”, according to some embodiments, refers to compounds and/or compositions that comprise at least 92% CBD or any functional derivative thereof. The phrase “CBD or any functional derivative thereof”, according to some embodiments, refers to compounds and/or compositions that comprise at least 95% CBD or any functional derivative thereof. The phrase “CBD or any functional derivative thereof”, according to some embodiments, refers to compounds and/or compositions that comprise at least 97% CBD or any functional derivative thereof. The phrase “CBD or any functional derivative thereof”, according to some embodiments, refers to compounds and/or compositions that comprise at least 99% CBD or any functional derivative thereof.

In one embodiment, substantially and/or essentially devoid of THC is less than 10% by weight or weight/weight THC. In one embodiment, substantially and/or essentially devoid of THC is less than 7% by weight or weight/weight THC. In one embodiment, substantially and/or essentially devoid of THC is less than 5% by weight or weight/weight THC. In one embodiment, substantially and/or essentially devoid of THC is less than 3% by weight or weight/weight THC. In one embodiment, substantially and/or essentially devoid of THC is less than 1% by weight or weight/weight THC. In one embodiment, substantially and/or essentially devoid of THC is less than 0.5% by weight or weight/weight THC. In one embodiment, substantially and/or essentially devoid of THC is less than 0.3% by weight or weight/weight THC. In one embodiment, substantially and/or essentially devoid of THC is less than 0.1% by weight or weight/weight THC.

Cannabis sativa, also termed marijuana, produces a variety of Cannabinoids, including Δ⁹-tetrahydrocannabinol (THC) and Cannabidiol (CBD). The term “cannabinoids” refers to a heterogeneous family of molecules usually exhibiting pharmacological properties by interacting with specific receptors. To date, two membrane receptors for cannabinoids, both coupled to G protein and named CB1 and CB2 have been identified. While CB1 receptors are mainly expressed in the central and peripheral nervous system, CB2 receptors have been reported to be more abundantly detected in cells of the immune system.

THC is the main psychoactive Cannabinoid in Cannabis, and while it is used as a treatment for a number of medical conditions, its use is strongly limited by the unavoidable psychotropic effects.

In the methods described herein, cannabidiol, or a functional variant thereof, is administered to a subject suffering from severe forms of acute and/or chronic GVHD. In the methods described herein, cannabidiol, or a functional variant thereof, free or substantially free of THC, is administered to a subject suffering from severe forms of acute and/or chronic GVHD. In the methods described herein, purified or substantially purified (greater than 80% w/w, 85% w/w, 90%, w/w 95% w/w or 97% w/w) cannabidiol, or a functional variant thereof, is administered to a subject suffering from acute and/or chronic GVHD. Cannabidiol constitutes up to 40% of Cannabis sativa extracts, and is recognized as a major non-psychoactive cannabinoid, with a remarkable lack of any cognitive and psychoactive actions. CBD, also termed 2-[(6R)-3-Methyl-6-prop-1-en-2-yl-1cyclohex-2-envyl]-5pentylbenzene-1,3-diol, has the molecular formula of C21H3002. The chemical structure of CBD is shown in Formula I:

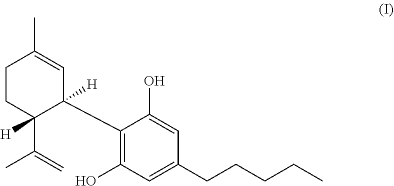 [SMILES: C=C(C)[C@@H]1CCC(C)=C[C@H]1c1c(O)cc(CCCCC)cc1O]

Cannabidiol is insoluble in water but soluble in organic solvents, such as oil. Accordingly, CBD can be formulated for use in the described methods through use of any organic solvent known to the pharmaceutical arts, including, but not limited to edible oils. When formulated for oral administration, any edible oil can be used in the CBD formulation, including olive oil.

A CBD comprises CBD derivative, which is in some embodiments, a metabolite of CBD such as but not limited to: (−)-7-hydroxy-CBD and (−)-CBD-7-oic acid and their dimethylheptyl (DMH) homologs, as well as of the corresponding compounds in the enantiomeric (+)-CBD series. A CBD derivative is characterized, in some embodiments, by a structure wherein at least one of the hydroxyl substituent groups is converted to a stable form thereof. In one embodiment, a CBD derivative is cannabinol comprising a quinone ring. In one embodiment, a CBD derivative is an endocannabinoid derivative. In some embodiments, a CBD derivative is described in Frank D King; G Lawton; A W Oxford Progress in medicinal chemistry. Vol. 44. Pages 207-331, Elsevier Science, 2006 ISBN: 0080462103 9780080462103 which is hereby incorporated by reference in its entirety.

The safety of Cannabidiol has been shown in several clinical studies performed in patient suffering from psychological or neurological disorders. The reports indicate that administration of Cannabidiol in doses of up to 1500 mg per day resulted without any significant adverse effects. In some embodiment, the dose, dosage, or daily dose or daily dosage of Cannabidiol or a functional derivative thereof is 50 to 2500 mg. In some embodiment, the dose, dosage, or daily dose or daily dosage of Cannabidiol or a functional derivative thereof is 150 to 1500 mg. In some embodiment, the dose, dosage, or daily dose or daily dosage of Cannabidiol or a functional derivative thereof is 100 to 1000 mg. In some embodiment, the dose, dosage, or daily dose or dosage of Cannabidiol or a functional derivative thereof is 200 to 1000 mg. In some embodiment, the dose, dosage, or daily dose or daily dosage of Cannabidiol or a functional derivative thereof is the therapeutically effective dose. In one embodiment, a dose is a single dose or a daily dose. In one embodiment, a dose is multiple daily doses. In one embodiment, a dose is administered twice or 3 times daily.

The methods of the present invention provide long desired therapy of acute and chronic GVHD by inhibiting the destructive immunologic process, alleviate symptoms associated with GVHD and/or steroidal therapy, and prevent disease progression. Principal components of GVHD therapy, according to this invention, may include systemic treatment with immunosuppressive drugs or immune modulators integrated with ancillary therapy (non-systemic treatment directed to control the symptoms such as topical corticosteroids, and cyclosporine eye drops) and supportive care (interventions that are directed at control of organ-specific or systemic symptoms such as antibiotics for prevention of infections and physical therapy) and a therapeutic composition comprising Cannabidiol or a functional derivative thereof. The methods for treating acute and chronic GVHD of the present invention provide, in some embodiments, treatment with Cannabidiol or a functional derivative thereof combined with systemic treatment of prednisone, optionally combined with cyclosporine and/or tacrolimus. In some embodiments, Cannabidiol or a functional derivative thereof inhibit and/or decrease side-effects of a steroid, prednisone, tacrolimus, cyclosporine, or any combination thereof.

In some embodiments, CBD as described herein alleviates symptoms associated with both acute and chronic GVHD in transplanted patients.

The present invention now demonstrates the beneficial effects of treatment with Cannabidiol or a functional derivative thereof for the prevention or treatment of acute and chronic GVHD in transplanted patients. More specifically, the present invention demonstrates that treatment with the Cannabidiol or a functional derivative thereof compositions of the invention significantly reduces the incidence of acute and chronic GVHD and its severity, as well as other complications associated with the disease such as infections.

In some embodiments, the methods described herein are for treatment of acute GVHD, such as severe acute GVHD, for example Grade III or Grade IV GVHD. The acute GVHD for treatment by the described methods is in some embodiments be refractory to the currently-used therapies such as but not limited to: steroidal GVHD therapy. In other embodiments, CBD-inclusive treatments replace or accompany currently-used first-line acute GVHD treatments.

In one embodiment, provided a method for treating a subject afflicted with acute graft versus host disease (GVHD) or chronic GVHD comprising administering to the subject a therapeutically effective amount of a composition comprising a cannabidiol (CBD) or a functional derivative thereof, thereby treating a subject afflicted with acute graft versus host disease (GVHD) or chronic GVHD.

In one embodiment, GVHD is a steroid-refractory GVHD. In one embodiment, steroid-refractory GVHD is defined in: H. Joachim Deeg. How I treat refractory acute GVHD. Blood. 2007 May 15; 109(10): 4119-412 (PMCID: PMC1885485), S. Arai, J. Margolis, M. Zahurak, V. Anders, and G. B. Vogelsang, “Poor outcome in steroid-refractory graft-versus-host disease with antithymocyte globulin treatment,” Biology of Blood and Marrow Transplantation, vol. 8, no. 3, pp. 155-160, 2002, Jason R. Westin, Rima M. Saliba, Marcos De Lima, Amin Alousi, Chitra Hosing, Muzaffar H. Qazilbash, 1 Issa F. Khouri, Elizabeth J. Shpall, Paolo Anderlini, Gabriela Rondon, Borje S. Andersson, Richard Champlin, and Daniel R. Couriel. Steroid-Refractory Acute GVHD: Predictors and Outcomes. Advances in Hematology. Volume 2011 (2011) pages 1-8, Article ID 601953, all of which are incorporated by reference in their entirety.

In one embodiment, acute GVHD is steroid-refractory GVHD. In one embodiment, acute GVHD is steroid-refractory GVHD. In one embodiment, a subject suffering from steroid-refractory GVHD will not respond (GVHD and associated side effects are not inhibited and/or treated) to methylprednisolone (MP), 0.5 to 20 mg/kg per day for 2 to 30 days. In one embodiment, a subject suffering from steroid-refractory GVHD will not respond (GVHD and associated side effects are not inhibited and/or treated) to MP, 2 to 15 mg/kg per day for 2 to 30 days. In one embodiment, a subject suffering from steroid-refractory GVHD will not respond (GVHD and associated side effects are not inhibited and/or treated) to methylprednisolone (MP), 2 to 12 mg/kg per day for 2 to 30 days.

In one embodiment, 2 to 30 days is 2 to 5 days. In one embodiment, 2 to 30 days is 2 to 5 days. In one embodiment, 2 to 30 days is 3 to 15 days. In one embodiment, 2 to 30 days is 3 to 6 days. In one embodiment, 2 to 30 days is 3 to 14 days. In one embodiment, 2 to 30 days is 10 to 30 days. In one embodiment, 2 to 30 days is 12 to 25 days. In one embodiment, 2 to 30 days is 3 to 25 days.

In one embodiment, a subject suffering from steroid-refractory GVHD will not respond (GVHD and associated side effects are not inhibited and/or treated) to a steroid treatment of 0.5 to 25 mg/kg per day for 2 to 30 days. In one embodiment, a subject suffering from steroid-refractory GVHD will not respond (GVHD and associated side effects are not inhibited and/or treated) to a combined steroid treatment of 0.5 to 25 mg/kg per day for 2 to 30 days. In one embodiment, treatment of GVHD includes inhibiting the progression from grade IIa to grade IIb and/or from grade IIb to grade III and/or from grade III to grade IV. In one embodiment, steroid-refractory GVHD may be characterized as GVHD wherein steroid treatment fails to inhibit the progression from grade IIa to grade IIb and/or from grade IIb to grade III and/or from grade III to grade IV. In one embodiment, treatment of GVHD includes inhibiting the progression from grade IIa, grade IIb and/or grade III to grade IV. In one embodiment, treatment of GVHD includes inhibiting the progression from grade IIa to grade III. In one embodiment, treatment of GVHD includes inhibiting the progression from a grade lower than IV to grade IV.

In one embodiment, a steroid is a corticosteroid and/or glucocorticoid or a combination of glucocorticoids and/or corticosteroids. In one embodiment, the steroid is methylprednisolone, prednisolone, hydrocortisone, dexamethasone, beclomethasone, budesonide, clobetasol, triamcinolone, fluticasone, mometasone, Diflorasone Desoximetasone, aclometasone, fluocinonide, halobetasol, flurandrenolide, betamethasone, cortisone, prednisone or any equivalents thereof and/or a combination thereof.

In one embodiment, steroid daily dose is a single dose per day. In one embodiment, steroid daily dose is divided to 2 to 8 portions/doses per day. In one embodiment, steroid treatment includes 0.2 to 25 mg/kg (body weight) per day steroid, for 2 to 30 days.

In one embodiment, a subject as described herein is a human. In one embodiment, a subject as described cannot benefit from steroid treatment. In one embodiment, a steroid treatment is refractory to a subject such as described herein.

Treating, according to some embodiments, includes inhibiting the progression or deterioration of GVHD. In one embodiment, inhibiting the progression of GVHD is inhibiting the progression from grade II to grade III GVHD. In one embodiment, inhibiting the progression of GVHD is inhibiting the progression from grade III to grade IV GVHD. In one embodiment, inhibiting the progression of GVHD is reducing the risk of death in grade III and grade IV GVHD patients. In another embodiment, grade III and grade IV GVHD are defined as acute GVHD. In another embodiment, acute GVHD is a deteriorating or progressing GVHD.

In one embodiment, a therapeutically effective amount of a composition comprising a CBD or a functional derivative thereof comprises 0.5 mg to 1 g of a CBD or a functional derivative thereof. In one embodiment, a therapeutically effective daily dose of a CBD or a functional derivative thereof comprises 0.5 mg to 1 g of a CBD or a functional derivative thereof. In one embodiment, a therapeutically effective amount of a composition comprising a CBD or a functional derivative thereof comprises 5 mg to 750 mg of a CBD or a functional derivative thereof. In one embodiment, a therapeutically effective daily dose of a CBD or a functional derivative thereof comprises 5 mg to 750 mg of a CBD or a functional derivative thereof. In one embodiment, a therapeutically effective amount of a composition comprising a CBD or a functional derivative thereof comprises 5 mg to 600 mg of a CBD or a functional derivative thereof. In one embodiment, a therapeutically effective daily dose of a CBD or a functional derivative thereof comprises 5 mg to 600 mg of a CBD or a functional derivative thereof. In one embodiment, a therapeutically effective amount of a composition comprising a CBD or a functional derivative thereof comprises 50 mg to 500 mg of a CBD or a functional derivative thereof. In one embodiment, a therapeutically effective daily dose of a CBD or a functional derivative thereof comprises 50 mg to 500 mg of a CBD or a functional derivative thereof. In one embodiment, a therapeutically effective amount of a composition comprising a CBD or a functional derivative thereof comprises 80 mg to 400 mg of a CBD or a functional derivative thereof. In one embodiment, a therapeutically effective daily dose of a CBD or a functional derivative thereof comprises 80 mg to 400 mg of a CBD or a functional derivative thereof. In one embodiment, a therapeutically effective daily dose of a CBD or a functional derivative thereof comprises 80 mg to 600 mg of a CBD or a functional derivative thereof. In one embodiment, a single therapeutically effective dosage of CBD or a functional derivative thereof comprises 30 mg to 400 mg of a CBD or a functional derivative thereof. In one embodiment, a single therapeutically effective dosage of CBD or a functional derivative thereof comprises 50 mg to 500 mg of a CBD or a functional derivative thereof. In one embodiment, a single therapeutically effective dosage of CBD or a functional derivative thereof comprises 80 mg to 300 mg of a CBD or a functional derivative thereof. In one embodiment, a single therapeutically effective dosage of CBD or a functional derivative thereof comprises 100 mg to 200 mg of a CBD or a functional derivative thereof.

In one embodiment, a composition as described herein is a topical composition. In one embodiment, a composition as described herein is an oral composition. In one embodiment, a composition as described herein is a systemic composition. In one embodiment, a subject as described herein is treated with a combination of steroid compositions selected from: a topical composition, a systemic composition, and an oral composition.

In one embodiment, the method further includes administering at least one additional GVHD therapeutic agent. In one embodiment, an additional GVHD therapeutic agent is: Antithymocyte globulin, Alemtuzumab, an Interleukin-2 receptor antagonist, a steroid, an antitumour necrosis factor antibody, Mycophenolate mofetil, Sirolimus, Pentostatin, Mesenchymal stem cells, or any combination thereof. In some embodiments, a subject as described herein is treated with a steroid prior to the treatment with a CBD or a functional derivative thereof as described herein. In some embodiments, a subject as described herein is treated with CBD or a functional derivative thereof and a steroid. In some embodiments, a subject as described herein is treated with CBD or a functional derivative thereof and at least one additional GVHD therapeutic agent.

Conditions Associated with GVHD

In one embodiment, the invention provides a method for treating a subject afflicted with a condition associated with acute graft versus host disease (GVHD) or chronic GVHD, comprising administering to the subject a therapeutically effective amount of a composition comprising a cannabidiol (CBD) or a functional derivative thereof, thereby treating a subject afflicted with a condition associated with acute graft versus host disease (GVHD).

In one embodiment, a condition associated with acute graft versus host disease (GVHD) or chronic GVHD is selected from the group consisting of: maculopapular rash, generalized erythroderma and bullous formation, hyperbilirubinemia, diarrhea, nausea, scleroderma, or any combination thereof. In one embodiment, a condition associated with acute graft versus host disease (GVHD) or chronic GVHD comprises a side effect resulting from a steroid treatment. In one embodiment, a condition associated with acute graft versus host disease (GVHD) or chronic GVHD comprises cholestatic hyperbilirubinemia. In one embodiment, a condition associated with acute graft versus host disease (GVHD) or chronic GVHD comprises cholestatic hyperbilirubinemia. In one embodiment, provided herein a method for treating a subject afflicted with GVHD and hyperbilirubinemia. In one embodiment, provided herein a method for reducing bilirubin blood level in a subject afflicted with GVHD and hyperbilirubinemia. In one embodiment, provided herein a method for reducing bilirubin blood level by at least 10% in a subject afflicted with GVHD and hyperbilirubinemia. In one embodiment, provided herein a method for reducing bilirubin blood level by at least 20% in a subject afflicted with GVHD and hyperbilirubinemia. In one embodiment, provided herein a method for reducing bilirubin blood level by at least 30% in a subject afflicted with GVHD and hyperbilirubinemia. In one embodiment, provided herein a method for reducing bilirubin blood level by at least 40% in a subject afflicted with GVHD and hyperbilirubinemia. In one embodiment, provided herein a method for reducing bilirubin blood level by at least 50% in a subject afflicted with GVHD and hyperbilirubinemia. In one embodiment, provided herein a method for reducing bilirubin blood level by at least 60% in a subject afflicted with GVHD and hyperbilirubinemia.

In one embodiment, treating a subject afflicted with a condition associated with acute graft versus host disease (GVHD) or chronic GVHD is reducing the severity of a condition associated with acute graft versus host disease (GVHD) or chronic GVHD. In one embodiment, treating a subject afflicted with a condition associated with acute graft versus host disease (GVHD) or chronic GVHD is inhibiting and/or ameliorating a condition associated with acute graft versus host disease (GVHD) or chronic GVHD.

In one embodiment, acute GVHD is acute skin GVHD grade III or grade IV. In one embodiment, acute GVHD is acute liver GVHD grade III or grade IV. In one embodiment, acute GVHD is acute gastrointestinal GVHD grade III or grade IV. In one embodiment, acute liver GVHD grade III or grade IV includes hyperbilirubinemia.

In one embodiment, provided a method for enhancing the therapeutic effect of a steroid in a subject in need of a steroid therapy, comprising administering to the subject the steroid and a cannabidiol (CBD) or a functional derivative thereof, thereby enhancing the therapeutic effect of a steroid in a subject in need of a steroid therapy. In one embodiment, provided a method for enhancing the therapeutic effect of a steroid dose or dosage in a subject in need of a steroid therapy, comprising administering to the subject the steroid dose or dosage and a cannabidiol (CBD) or a functional derivative thereof, thereby enhancing the therapeutic effect of a steroid dose or dosage in a subject in need of a steroid therapy. In one embodiment, enhancing the therapeutic effect of a steroid is maintaining a fixed dose and combining it with a cannabidiol (CBD) or a functional derivative thereof. In one embodiment, enhancing the therapeutic effect of a steroid is rendering a refractory steroid dose and/or dosage, therapeutically effective. In one embodiment, enhancing the therapeutic effect of a steroid is rendering a sub-efficient steroid dose and/or dosage, therapeutically effective. In one embodiment, enhancing the therapeutic effect of a steroid is avoiding increase in steroid dose and/or dosage. In one embodiment, enhancing the therapeutic effect of a steroid is avoiding increase in steroid dose and/or dosage due to insufficient and/or poor clinical effect. In one embodiment, enhancing the therapeutic effect of a steroid is reducing the duration of steroid treatment. In one embodiment, a steroid is a Glucocorticoid (glucocorticosteroid), corticosteroid or any steroid known to one of skill in the art or described herein.

In one embodiment, provided a pharmaceutical composition comprising a cannabidiol (CBD) or a functional derivative thereof for use in enhancing the therapeutic effect of a steroid. In one embodiment, provided a pharmaceutical composition comprising a cannabidiol (CBD) or a functional derivative thereof for use in reducing a dose or a dosage of a steroid. In one embodiment, provided a pharmaceutical composition comprising a cannabidiol (CBD) or a functional derivative thereof for use in reducing a dose or a dosage of a steroid while maintaining or enhancing the steroid's therapeutic effect. In one embodiment, provided a pharmaceutical composition comprising a cannabidiol (CBD) or a functional derivative thereof for use in maintaining or enhancing the therapeutic effect of a steroid therapy. In one embodiment, maintaining or enhancing the therapeutic effect of a steroid therapy, according to the methods of the invention, include reducing the dosage or dose of a steroid in a subject treated with a cannabidiol (CBD) or a functional derivative thereof.

In one embodiment, provided a pharmaceutical composition comprising a cannabidiol (CBD) or a functional derivative thereof for reducing the effective dose of a steroid. In one embodiment, provided a pharmaceutical composition comprising a cannabidiol (CBD) or a functional derivative thereof for reducing a side effect associated with steroid treatment. In one embodiment, provided a pharmaceutical composition comprising a cannabidiol (CBD) or a functional derivative thereof for reducing the effective dose of a steroid and thereby reducing a side effect associated with steroid treatment.

In one embodiment, provided a pharmaceutical composition comprising a cannabidiol (CBD) or a functional derivative thereof for reducing the effective dose of a steroid. In one embodiment, “effective dose” is the “therapeutically effective dose”. In one embodiment, a pharmaceutical composition comprising a cannabidiol (CBD) or a functional derivative thereof reduces the effective dose of steroid by at least 20% w/w. In one embodiment, a pharmaceutical composition comprising a cannabidiol (CBD) or a functional derivative thereof reduces the effective dose of steroid by 10% to 70% w/w. In one embodiment, a pharmaceutical composition comprising a cannabidiol (CBD) or a functional derivative thereof reduces the effective dose of steroid by at least 30% w/w. In one embodiment, a pharmaceutical composition comprising a cannabidiol (CBD) or a functional derivative thereof reduces the effective dose of steroid by at least 40% w/w. In one embodiment, a pharmaceutical composition comprising a cannabidiol (CBD) or a functional derivative thereof reduces the effective dose of steroid by at least 50% w/w. In one embodiment, a pharmaceutical composition comprising a cannabidiol (CBD) or a functional derivative thereof reduces the effective dose of steroid by at least 60% w/w.

In one embodiment, provided a method for enhancing the therapeutic effect of a steroid in a subject afflicted with graft versus host disease (GVHD), comprising administering to the subject the steroid and a cannabidiol (CBD) or a functional derivative thereof, thereby enhancing the therapeutic effect of a steroid in a subject afflicted with graft versus host disease (GVHD). In one embodiment, GVHD is chronic GVHD. In one embodiment, GVHD is acute GVHD.

In one embodiment, provided a method for reducing a side effect associated with a steroid in a subject afflicted with graft versus host disease (GVHD) and treated with a steroid, comprising administering to the subject the steroid and a cannabidiol (CBD) or a functional derivative thereof. In one embodiment, provided a method for reducing a side effect associated with a steroid in a subject afflicted with graft versus host disease (GVHD) and treated with a steroid, comprising administering to the subject the steroid and a cannabidiol (CBD) or a functional derivative thereof. In one embodiment, provided a method for reducing the effective dose of a steroid in a subject afflicted with graft versus host disease (GVHD) and treated with a steroid, comprising administering to the subject a reduced steroid dose and a cannabidiol (CBD) or a functional derivative thereof.

In one embodiment, provided a method for enhancing the therapeutic effect of a steroid or reducing the effective dose of the steroid in a subject afflicted with acute respiratory distress syndrome (ARDS), inflammatory bowel disease (IBD) such as Crohn's disease, or Urticaria including: chronic Urticaria, Chronic Spontaneous Urticaria, or acute Urticaria, comprising administering to the subject the steroid and cannabidiol (CBD), thereby enhancing the therapeutic effect of the steroid in the subject afflicted with COVID-19, IBD, or Urticaria.

In one embodiment, provided herein is a method for reducing the effective dose of antihistamines in a chronic disease controllable or treatable by an antihistamine, comprising administrating a combination of CBD and an of antihistamine. In one embodiment, provided herein is a method for reducing the effective dose of an antihistamine in a chronic disease controllable or treatable by an antihistamine, comprising administrating a combination of CBD and a sub-effective dose of antihistamine. In one embodiment, provided herein is a method for reducing the effective dose of an antihistamine in a subject afflicted with Urticaria, comprising administering CBD and antihistamine. In one embodiment, provided herein is a method for reducing the effective dose of antihistamines in a subject afflicted with Urticaria, comprising administering CBD and a sub-effective dose of antihistamine.

In one embodiment, provided herein is a method for reducing the effective dose of an antihistamine and a steroid in a chronic disease controllable or treatable by an antihistamine and a steroid, comprising administrating a combination of CBD, steroid and antihistamine. In one embodiment, provided herein is a method for reducing the effective dose of an antihistamine and a steroid in a chronic disease controllable or treatable by an antihistamine and a steroid, comprising administrating a combination of CBD, a sub-effective dose of a steroid and a sub-effective dose of an antihistamine. In one embodiment, a chronic disease controllable or treatable by an antihistamine and a steroid is Urticaria, chronic Urticaria, allergic rhinitis, conjunctivitis allergic, atopic dermatitis (AD), anaphylaxis, nonallergic angioedema, otitis media, sinusitis, insomnia, anxiety, asthma, migraine and other vestibular disorders.

In one embodiment, enhancing the therapeutic effect of the steroid comprises: reducing a side effect associated with the steroid, reducing the dose of the known effective dose and maintaining the effectiveness of steroid treatment or rendering a refractory steroid dose a therapeutic effective dose. In one embodiment, enhancing the therapeutic effect of the steroid comprises setting a new effective dose of a steroid which is lower than the known or required effective dose of the steroid. In one embodiment, co-administration of CBD and steroid enables setting a new effective dose of the steroid which is lower than the known or required (without the administration of CBD) effective dose of the steroid. In one embodiment, enhancing the therapeutic effect of the steroid is reducing the dose required for the effective dose of the steroid.

In one embodiment, enhancing the therapeutic effect of the steroid is enhancing or speeding the recovery of a subject afflicted with ARDS or reducing death risk or death rate in subject/s afflicted with said ARDS. In one embodiment, ARDS is COVID-19 or corona infection. In one embodiment, COVID-19 affected subject is a subject which developed or at risk of developing moderate to severe illness. In one embodiment, COVID-19 affected subject is in need of hospitalization. In one embodiment, the COVID-19 affected subject is treated with respiratory support, ventilatory support or a combination thereof.

In one embodiment, provided herein a method of tapering off steroid therapy in a subject afflicted with a chronic inflammatory disease, comprising the following consecutive steps: (a) administering to the subject CBD and effective dose of the steroid; (b) gradually decreasing the dose of the steroid and maintaining co-administration of CBD, wherein the rate of decreasing steroid dose is matched to maintain control of the chronic inflammatory disease; and (c) tapering off the steroid therapy, once the chronic inflammatory disease is controlled, inhibited, treated or ameliorated, while administering CBD. In one embodiment, a subject afflicted with a chronic inflammatory disease is previously treated with a steroid but not CBD and steroid. In one embodiment, inhibited comprises inhibiting flares, pain, and/or swelling. In one embodiment, tapering off steroid therapy is ceasing steroid therapy and administering CBD. In one embodiment, tapering off steroid therapy administering CBD but not the steroid. In one embodiment, the result of tapering off steroid therapy provides a clinical control of a disease with reduced or no steroid therapy which is equivalent to the level of clinical control of a disease with the known, established, effective steroid of the in a subject afflicted with a chronic inflammatory disease as described herein.

In one embodiment, the chronic inflammatory disease is an inflammatory bowel disease (IBD) or Urticaria. In one embodiment, IBD is Crohn's disease. In one embodiment, IBD is ulcerative colitis. In one embodiment, the subject destined to tapering off steroid therapy as described herein is afflicted with a chronic inflammatory disease which is controlled, inhibited, treated or ameliorated by the effective dose of the steroid. In one embodiment, the subject destined to tapering off steroid therapy as described herein is afflicted with a chronic inflammatory disease which is controlled, inhibited, treated or ameliorated by the effective dose of the steroid and is not treated with CBD. In one embodiment, the chronic inflammatory disease in the subject destined to tapering off steroid therapy, is controlled, inhibited, treated, or ameliorated by the effective dose of the steroid. (the subject is initially (prior to employing the tapering off steroid therapy is treated with a steroid but not CBD)). In one embodiment, CBD dose in step (b) is increased concomitantly with decreasing the dose of the steroid.

In one embodiment, a steroid is an anabolic steroid. In one embodiment, a steroid is a corticosteroid. In one embodiment, a subject in need of a steroid therapy is afflicted with inflammation. In one embodiment, a subject in need of a steroid therapy is in need of reducing and/or inhibiting an immune response. In one embodiment, a subject in need of a steroid therapy is afflicted with a disease characterized by cytokine storm. In one embodiment, a subject in need of a steroid therapy is afflicted with a disease characterized by cytokine storm. In one embodiment, a subject in need of a steroid therapy is afflicted with Urticaria. In one embodiment, a subject in need of a steroid therapy is afflicted with chronic Urticaria. In one embodiment, a subject in need of a steroid therapy is afflicted with acute Urticaria. In one embodiment, a subject in need of a steroid therapy is afflicted with COVID-19. In one embodiment, a subject in need of a steroid therapy is afflicted with inflammatory bowel disease (IBD) including but not limited to Crohn's disease. In one embodiment, Crohn's disease is steroid dependent Crohn's Disease. In one embodiment, inflammatory bowel disease (IBD) is steroid dependent IBD.

Thus, in one aspect, the invention relates to a method of preventing, treating, ameliorating, or curing Urticaria. In one embodiment, the invention relates to a method of preventing, treating, ameliorating, or curing ARDS (COVID-19). In one embodiment, the invention relates to a method of preventing, treating, ameliorating, or curing IBD including but not limited to Crohn's disease. In one embodiment, preventing or treating comprises inhibiting the deterioration of the disease. In one embodiment, preventing or treating comprises inhibiting the severity of the disease.

In one embodiment, the invention relates to a method of preventing, treating, ameliorating, or curing a disease such as described herein by rendering an ineffective dose of a steroid, an effective dose. In one embodiment, the invention relates to a method of: (a) selecting a subject afflicted with a disease as described herein and at risk of a steroid therapy; and (b) treating the subject with CBD or a combination of CBD and steroid.

In one embodiment, the invention relates to a method of preventing, treating, ameliorating, or curing a disease such as described herein by rendering an ineffective dose of a steroid, an effective dose. In one embodiment, the invention relates to a method of: (a) selecting a subject afflicted with a disease as described herein and refractory to a steroid therapy; and (b) treating the subject with CBD or a combination of CBD and steroid. In one embodiment, the disease itself as described herein is refractory to prolonged systemic treatment of steroids.

In one embodiment, the invention relates to a method of controlling a disease such as described with a steroid dose lower than the effective dose for controlling the disease, by administering CBD as described. In one embodiment, the invention relates to a method of preventing flares in a disease such as described with a steroid dose that is lower than the effective dose for preventing flares, by administering CBD as described. In one embodiment, the invention relates to a method of enhancing the efficacy of a steroid by co-administering CBD with the steroid. In one embodiment, the invention relates to a method of gradually reducing the dose of a steroid by co-administering CBD with the steroid. In one embodiment, controlling is inhibiting flares. In one embodiment, controlling is inhibiting a cytokine storm. In one embodiment, controlling is inhibiting inflammation.

In one embodiment, the invention relates to a method of treating a subject afflicted with IBD including but not limited to Crohn's disease, comprising reducing the effective steroid dose without inducing flares by co-administering CBD with the reduced or sub-effective steroid dose. In one embodiment, the invention relates to a method of treating a subject afflicted with IBD including but not limited to Crohn's disease, comprising reducing the effective steroid dose without inducing flares and without addition of IBD or Crohn's Disease specific drugs, by co-administering CBD with the reduced or sub-effective steroid dose.

In one embodiment, the disease is chronic, acute or both. In one embodiment, the disease is steroid dependent. In one embodiment, steroid administration as described herein is chronic or long term (3 weeks, 1 month, 3 months, 6 months, 1 year, 2 years, 5 years, 10 years or any duration therebetween) administration.

In one embodiment, the invention relates to a method of treating a subject afflicted with a disease or a chronic disease such as described herein, wherein the subject is treated with a steroid comprising administering to the subject CBD. In one embodiment, the invention relates to a method of treating a subject afflicted with a disease or a chronic disease such as described herein, wherein the subject is treated with a steroid dose controlling the disease as described herein comprising administering to the subject CBD. In one embodiment, the invention relates to a method of treating a subject afflicted with a disease or a chronic disease such as described herein, wherein the subject is treated with a steroid dose controlling the disease as described herein comprising administering to the subject CBD and thereafter decreasing the steroid dose.

In one embodiment, the invention relates to a method of treating a subject afflicted with a disease or a chronic disease such as described herein, wherein the subject is treated with a steroid dose controlling the disease as described herein comprising administering to the subject CBD and decreasing the steroid dose. In one embodiment, the invention relates to a method of treating a subject afflicted with a disease or a chronic disease such as described herein, wherein the subject is treated with a steroid dose controlling the disease as described herein comprising administering to the subject CBD and decreasing the steroid dose. In one embodiment, a method of treating a subject afflicted with a disease or a chronic disease such as described herein comprises controlling the disease as described herein. In one embodiment controlling the disease or the chronic disease as described herein with a reduced or sub-effective dose of a steroid is feasible or conceivable by administering CBD with the reduced or sub-effective dose of a steroid.

In one embodiment, provided herein a method of replacing steroid therapy with CBD therapy while controlling, treating, inhibiting or ameliorating a disease as described herein, comprising the step of co-administering an effective steroid dose and CBD followed by gradually decreasing the dose of the steroid until the disease is controlled, inhibited, treated or ameliorated by administering CBD but not the steroid.

In one embodiment, provided herein a method for enhancing the recovery or reducing death risk of a subject afflicted with a disease as described herein comprising the step of administering to the subject a steroid and CBD. In one embodiment, provided herein a method for enhancing the recovery or reducing death risk of a subject afflicted with Acute respiratory distress syndrome (ARDS) such as but not limited to COVID-19 comprising the step of administering to the subject a combined therapy of a steroid and CBD. In one embodiment, provided herein a method for enhancing the recovery or reducing death risk of a subject afflicted with ARDS such as but not limited to COVID-19 and in need of respiratory support and/or ventilatory support comprising the step of administering to the subject a combined therapy of a steroid and CBD. In one embodiment, “enhancing the recovery” comprises shortening the hospitalization period of the subject.

In one embodiment, provided herein a method of tapering off steroid therapy with CBD therapy while controlling, treating, inhibiting, or ameliorating a disease as described herein, comprising the step of co-administering an effective steroid dose and CBD followed by gradually decreasing the dose of the steroid until the disease is controlled, inhibited, treated or ameliorated by administering CBD but not the steroid. In one embodiment, provided herein a method of tapering off steroid therapy with CBD therapy while controlling, treating, inhibiting, or ameliorating a chronic inflammatory disease as described herein, comprising the step of co-administering an effective steroid dose and CBD followed by gradually decreasing the dose of the steroid until the disease is controlled, inhibited, treated or ameliorated by administering CBD but not the steroid. In one embodiment, provided herein a method of tapering off steroid therapy with CBD therapy while controlling, treating, inhibiting, or ameliorating IBD including but not limited to Crohn's disease as described herein, comprising the step of co-administering an effective steroid dose and CBD followed by gradually decreasing the dose of the steroid until the disease is controlled, inhibited, treated or ameliorated by administering CBD but not the steroid. In one embodiment, tapering off is reducing steroid dose to a minimal and sub-effective dose or not administering any steroid.

In one embodiment, the invention relates to a method rendering a refractive steroid dose a therapeutically effective dose by administering CBD. In one embodiment, a subject is afflicted with a cytokine storm. In one embodiment, a subject is afflicted with Acute respiratory distress syndrome (ARDS) such as but not limited to COVID-19 and is at risk of developing a cytokine storm, explosive acute inflammatory response in the lung parenchyma, leading to alveolar oedema, decreased lung compliance, hypoxaemia or any combination thereof.

In one embodiment, the subject is in need of a high-dose steroid treatment. In one embodiment, the subject is selected as a subject in need of a high-dose steroid treatment. In one embodiment, a high-dose steroid treatment is more than 25-mg, 30-mg or 40-mg daily dose of Prednisone. In one embodiment, a high-dose steroid treatment is more than 15-mg, 20-mg or 25-mg daily dose of cortisone. In one embodiment, a high-dose steroid treatment is pulse steroid treatment. In one embodiment, the subject is in need of a prolonged or chronic steroid treatment. In one embodiment, the subject is in need of a prolonged steroid treatment lasting for at least 21 days, 4 weeks, 2 months, 3 months, 4 months, 6 months, a year, 2 years, 5 years, 10 years or any duration therebetween.

In one embodiment, the steroid, corticosteroid or glucocorticosteroid is administered systemically. In one embodiment, the steroid is administered locally and/or topically.

In some embodiments, a reduced dose includes at least 15% (by weight) less steroid. In some embodiments, a reduced dose includes at least 20% (by weight) less steroid. In some embodiments, a reduced dose includes at least 25% (by weight) less steroid. In some embodiments, a reduced dose includes at least 30% (by weight) less steroid. In some embodiments, a reduced dose includes at least 40% (by weight) less steroid. In some embodiments, a reduced dose includes at least 50% (by weight) less steroid.

In some embodiments, the phrase “effective dose” is the dose effective for treating or ameliorating GVHD as described herein. In some embodiments, the phrase “effective dose” is synonymous with the phrase “daily effective dose” as described herein. In some embodiments, the phrase “effective dose” is the dose effective for reducing bilirubin level in the blood of a subject afflicted with acute liver GVHD. In some embodiments, the phrase “effective dose” is the dose effective for reducing and/or inhibiting a pathology and/or risk associated with chronic or acute GVHD. In some embodiments, reducing the effective dose of a steroid results in minimizing or reducing at least one side effect associated with steroid treatment.

In one embodiment, provided a method for enhancing the therapeutic GVHD effect of a given steroid dose in a subject afflicted with graft versus host disease (GVHD) and treated with a steroid, comprising administering to the subject the given steroid dose and a cannabidiol (CBD) or a functional derivative thereof.

In some embodiments, enhancing the therapeutic effect of a steroid enables the reduction of dosing. In some embodiments, enhancing the therapeutic effect of a steroid is rendering a refractory steroid dose a therapeutic effective dose. In some embodiments, a refractory steroid dose is any steroid dose found to be refractory in a subject afflicted with GVHD. In one embodiment, a refractory steroid dose is a daily steroid dose of 0.2 to 20 mg/kg (body weight) per day found to be refractory in terms of therapeutic effect in a subject afflicted with GVHD. In one embodiment, provided a method for treating a subject afflicted with steroid-refractory GVHD, comprising administering to the subject: (a) a steroid; and (b) a cannabidiol (CBD) or a functional derivative thereof. In some embodiments, enhancing the therapeutic effect of a steroid is enhancing the therapeutic effect of a given or a fixed dose of a steroid. In some embodiments, enhancing the therapeutic effect of a steroid is enhancing steroid therapy which is reducing or gradually reducing (within a period of 3 days to 6 months) the administered dosage or dose of a steroid while maintain and/or improving/enhancing/maintaining: (a) the therapeutic effect of the reduced dose of a steroid; or (b) the efficacy of the steroid therapy. In some embodiments, enhancing the therapeutic effect of a steroid is rendering a refractory steroid therapy (treatment with a given steroid dose)—therapeutically effective and optionally further reducing or gradually reducing (within a period of 3 days to 6 months) the administered dosage or dose of a steroid (the dose or dosage found previously to be refractory) while continuously maintaining and/or improving/enhancing: (a) the therapeutic effect of the reduced dose of a steroid; or (b) the efficacy of the steroid therapy.

In one embodiment, the steroid is Methyprednisolone or prednisone and both the refractory steroid dose the therapeutic effective dose is 0.1 to 3 mg/kg/day. In one embodiment, the steroid is Methyprednisolone or prednisone and both the refractory steroid dose the therapeutic effective dose is 0.2 to 2 mg/kg/day. In one embodiment, the steroid is Methyprednisolone or prednisone and both the refractory steroid dose the therapeutic effective dose is 0.5 to 1.8 mg/kg/day.

In some embodiments, enhancing the therapeutic effect of a given dose of a steroid or reducing the given dose of a steroid by maintain or improving the steroid's therapeutic effect can be a process wherein the steroid dose administered with CBD is gradually decreased over time. In some embodiments, enhancing the therapeutic effect of a given dose of a steroid or reducing the given dose of a steroid by maintain or improving the steroid's therapeutic effect can be a process wherein the steroid dose administered with CBD is gradually decreased over a period of 3 days to 6 months. In some embodiments, enhancing the therapeutic effect of a given dose of a steroid or reducing the given dose of a steroid by maintain or improving the steroid's therapeutic effect can be a process wherein the steroid dose administered with CBD is gradually decreased over a period of 3 weeks to 3 months. over a period of 3 weeks to 2 months.

In other embodiments, the methods include treatment of the conditions associated with severe acute GVHD, including pathologies of the skin, liver, and gastrointestinal tract. Such conditions or symptoms include maculopapular rash, generalized erythroderma and bullous formation, greater than normal levels of bilirubin (cholestatic hyperbilirubinemia; for example, greater than 2 mg %), diarrhea, and nausea.

In still other embodiments, the described methods include treatment of severe chronic forms of GVHD, including, severe classing and overlap forms of chronic GVHD.

In some embodiments, the treatment of the severe chronic GVHD is treatment of one or more symptoms or conditions associated with chronic GVHD. In one embodiment, conditions associated with the chronic GVHD are known to one of skill in the art.

In other embodiments treatment with the Cannabidiol or a functional derivative thereof compositions of the invention treats GVHD associated symptoms and signs of the various organs and systems, including the skin, nails, mouth, eyes, female genitalia, gastrointestinal tract, liver, lungs, muscles, fascia and joints.

Thus, in one aspect, the invention relates to a method of preventing, treating, ameliorating or curing acute and chronic GVHD, including Grade III or Grade IV acute GVHD, moderate to severe chronic GVHD, and steroid-refractory forms of the diseases. The method of the invention comprises the step of administering to a subject in need thereof a therapeutically effective amount of Cannabidiol or any functional derivative thereof or any pharmaceutical composition comprising the same.

In some embodiments, the Cannabidiol or any functional derivative thereof according to the present invention is a natural product extracted and/or purified from Cannabis sativa. In other embodiments, the CBD or functional derivative thereof is a synthetic product. In still further embodiments, the CDB-containing composition is the Cannabis plant itself. Whenever reference is made herein to “Cannabis sativa” the same applies also to other Cannabis plants producing Cannabidiol, including Cannabis indica and Cannabis ruderalis. Cannabis sativa is referred to herein specifically, for the sake of brevity.

In some embodiments, Cannabidiol, or any derivative thereof according to the present invention, can be administered to patients before or after any type of allogeneic hematopoietic stem cell transplantation, including but not limited to sibling and unrelated-donor bone marrow or peripheral-blood stem cell transplantation, cord blood transplantation, and haploidentical bone marrow or peripheral-blood stem cell transplantation.

In some embodiments, the CBD, or a functional derivative thereof, is administered following onset of symptoms of the acute or chronic GVHD as described herein. In other embodiments, the CBD, or a functional derivative thereof, is administered after a diagnosis is made of the form of GVHD. In other embodiments, the CBD, or a functional derivative thereof, is administered with a steroid. In other embodiments, the CBD or a functional derivative thereof, is administered before and/or after a steroid.

Administration of the Cannabidiol compositions to a patient intended to undergo transplantation may start between 14 to 5 days prior to the medical procedure, more specifically 8 days before the transplant. Alternatively, treatment with the Cannabidiol compositions may begin between 1 to 30 days after transplantation, more specifically, 1 day after the transplant.

Administration of the Cannabidiol or a functional derivative thereof compositions to a subject as described herein lasts for at least 20 days post transplantation or post development of GVHD. Administration of the Cannabidiol or a functional derivative thereof compositions to a subject as described herein lasts for at least 30 days post transplantation or post development of GVHD. Administration of the Cannabidiol or a functional derivative thereof compositions to a subject as described herein lasts for at least 20 days post transplantation or post development of GVHD. Administration of the Cannabidiol or a functional derivative thereof compositions to a subject as described herein lasts for at least 40 days post transplantation or post development of GVHD. Administration of the Cannabidiol or a functional derivative thereof compositions to a subject as described herein lasts for at least 50 days post transplantation or post development of GVHD. Administration of the Cannabidiol or a functional derivative thereof compositions to a subject as described herein lasts for at least 60 days post transplantation or post development of GVHD. Administration of the Cannabidiol or a functional derivative thereof compositions to a subject as described herein lasts for at least 70 days post transplantation or post development of GVHD. Administration of the Cannabidiol or a functional derivative thereof compositions to a subject as described herein lasts for at least 80 days post transplantation or post development of GVHD. Administration of the Cannabidiol or a functional derivative thereof compositions to a subject as described herein lasts for at least 90 days post transplantation or post development of GVHD. Administration of the Cannabidiol or a functional derivative thereof compositions to a subject as described herein lasts for at least 100 days post transplantation or post development of GVHD.

Administration of the Cannabidiol or a functional derivative thereof compositions to a subject as described herein lasts for at least 20 days from the day Cannabidiol or a functional derivative thereof was administered for the first time. Administration of the Cannabidiol or a functional derivative thereof compositions to a subject as described herein lasts for at least 30 days from the day Cannabidiol or a functional derivative thereof was administered for the first time. Administration of the Cannabidiol or a functional derivative thereof compositions to a subject as described herein lasts for at least 20 days from the day Cannabidiol or a functional derivative thereof was administered for the first time. Administration of the Cannabidiol or a functional derivative thereof compositions to a subject as described herein lasts for at least 40 days from the day Cannabidiol or a functional derivative thereof was administered for the first time. Administration of the Cannabidiol or a functional derivative thereof compositions to a subject as described herein lasts for at least 50 days from the day Cannabidiol or a functional derivative thereof was administered for the first time. Administration of the Cannabidiol or a functional derivative thereof compositions to a subject as described herein lasts for at least 60 days from the day Cannabidiol or a functional derivative thereof was administered for the first time. Administration of the Cannabidiol or a functional derivative thereof compositions to a subject as described herein lasts for at least 70 days from the day Cannabidiol or a functional derivative thereof was administered for the first time. Administration of the Cannabidiol or a functional derivative thereof compositions to a subject as described herein lasts for at least 80 days from the day Cannabidiol or a functional derivative thereof was administered for the first time. Administration of the Cannabidiol or a functional derivative thereof compositions to a subject as described herein lasts for at least 90 days from the day Cannabidiol or a functional derivative thereof was administered for the first time. Administration of the Cannabidiol or a functional derivative thereof compositions to a subject as described herein lasts for at least 100 days from the day Cannabidiol or a functional derivative thereof was administered for the first time. The first time Cannabidiol or a functional derivative was administered, in some embodiment, is the day grade IIa, grade IIb, grade III or grade IV, was diagnosed.

In some embodiments, a GVHD patient as described herein (the “subject”) is pre-treated with a calcineurine inhibitor (cyclosporine or tacrolimus) with or without a steroid such as but not limited to prednisone. In some embodiments, a GVHD patient as described herein (the “subject”) is treated with CBD or a functional derivative thereof with a calcineurine inhibitor (cyclosporine or tacrolimus) with or without a steroid such as but not limited to prednisone. In some embodiments, a GVHD patient as described herein (the “subject”) is treated with CBD or a functional derivative thereof with fludarabine, busulfex, melphalan, ATG or any combination thereof. In some embodiments, a GVHD patient as described herein is refractory to tacrolimus, a systemic steroid, a topical steroid or any combination thereof. In some embodiments, a GVHD patient as described herein is treated with 150 to 500 mg CBD or a functional derivative thereof per day and 15 to 100 mg steroid (such as prednisone) per day.

In one embodiment, a subject is afflicted with grade 3 or 4 acute GVHD of the gastro-intestinal tract (GIT). In one embodiment, a subject is afflicted with grade 3 or 4 acute GVHD of the liver. In one embodiment, a subject is afflicted with grade 3 or 4 acute GVHD of the skin. In one embodiment, a subject is afflicted with GVHD is afflicted with an overlap syndrome (concomitant acute plus chronic GVHD features).

In some embodiments, a GVHD patient as described herein (the “subject”) is treated with CBD or a functional derivative thereof with mycophenolate mofetil. In some embodiments, a GVHD patient as described herein (the “subject”) is treated with CBD or a functional derivative thereof with extra-corporeal photopheresis (ECP). In some embodiments, a GVHD patient as described herein (the “subject”) if further afflicted with myopathy. In some embodiments, the methods described herein lower blood bilirubin level in a subject having GVHD, from above 1.4 mg % or above 1.2 milligrams per deciliter (blood) to normal level (0.7-1.3 mg % or 1.2 milligrams per deciliter (mg/dL) blood) within a week or within 1 to 5 days. In some embodiments, the methods described herein lower blood bilirubin level in a subject having GVHD, from above 3.0 mg % or above 3.0 milligrams per deciliter (blood) to normal level (0.7-1.3 mg % or 1.2 milligrams per deciliter (mg/dL) blood) within a week or within 1 to 5 days. In some embodiments, the methods described herein lower blood bilirubin level in a subject having GVHD, from above 4.0 mg % (blood) to normal level (0.7-1.3 mg % or 1.2 milligrams per deciliter (mg/dL) blood) within a week or within 1 to 5 days. In some embodiments, the methods described herein lower blood bilirubin level in a subject having GVHD, from above 6.0 mg % (blood) to normal level (0.7-1.3 mg % or 1.2 milligrams per deciliter (mg/dL) blood) within a week or within 1 to 5 days. In some embodiments, the methods described herein lower blood bilirubin level in a subject having GVHD, from 2 to 9 mg % (blood) to normal level (0.7-1.3 mg % or 1.2 milligrams per deciliter (mg/dL) blood) within a week or within 1 to 5 days.

In some embodiments, the method of the invention may optionally further comprise the step of administering at least one additional therapeutic agent, including currently used drugs given to transplanted patients, whether prior to the transplant, or afterwards. These additional therapeutic agents, specifically, any immunomodulatory agent or known medicament, may be either combined with Cannabidiol or may be administered separately in an additional separate step having an optional different mode of administration.

In some embodiments, the method optionally further comprises the step of administering at least one additional therapeutic agent including but not limited to currently available medicines e.g. cyclosporine, tacrolimus, methotrexate, mycophenolate mofetil, sirolimus, ATG, imatinib or other TKIs, azathioprine, pentostatin, thalidomide, retinoids, anti-CD20, anti-CD52, ECP, corticosteroids and mesenchymal stem cells.

The pharmaceutical compositions containing Cannabidiol according to the present invention can be administered for prophylactic and/or therapeutic treatments. In therapeutic application, compositions are administered to a patient already suffering from acute or chronic GVHD in an amount sufficient to cure or at least partially arrest the condition and its complications. An amount adequate to accomplish this is defined as a “therapeutically effective dose.” In prophylactic applications, compositions containing Cannabidiol are administered to a patient who is at risk of developing acute and/or chronic GVHD, i.e. a patient being before or after allogeneic transplantation. Such an amount is defined to be a “prophylactically effective dose”. Amounts effective for both prophylactic and therapeutic purposes will depend upon the risk to develop GVHD, the severity of the GVHD condition and the general state of the patient, but generally range from about 0.01 to about 10 mg/Kg body weight, specifically, about 0.5 to about 10 mg/Kg of Cannabidiol per day. Single or multiple administrations on a daily schedule can be carried out with dose levels being selected by the treating physician. It should be noted that doses of Cannabidiol can be elevated every day during the treatment period according to the clinical response of the patient, provided no significant drug related side effects present.

Additionally, the administration of Cannabidiol according to the invention, or pharmaceutical compositions comprising Cannabidiol, may be periodic, for example, the periodic administration may be twice daily, three times daily, or at least once daily for 2 days to 180 days, more preferably 90 to 180 days after transplantation for GVHD prevention and 2 days to 12 months (or longer as needed) for the treatment of GVHD following onset of symptoms or diagnosis.

In some embodiments, CBD is provided to a patient in once, twice, thrice or more doses per day. Specific embodiments of the invention relate to the use of typically two doses per day, each containing at least 10 mg Cannabidiol, but usually not more than a daily dose of 600 mg. In one embodiment, a daily dose comprises 150 to 400 mg CBD administered in one or two dosages.

In some embodiments, an exemplary concentration of Cannabidiol in oil, e.g. olive oil, effective for the prevention and/or treatment of GVHD, such as Grade III or Grade IV acute GVHD or severe chronic GVHD, may range typically between about 1% weight/volume and about 3% weight/volume, more specifically, 2.5% weight/volume.

It should be noted that the Cannabidiol compositions according to the present invention can be prepared in any type of oil, such as canola oil, olive oil, sunflower oil, soybean oil, corn oil, or paraffin oil.

The administration of pharmaceutical compositions comprising Cannabidiol or any derivative thereof according to the invention for the prevention, treatment, amelioration of GVHD in any form, may be any one of oral, sublingual, buccal, rectal, vaginal, topical, parenteral, intravenous, intramuscular, subcutaneous, intra-peritoneal or via oral or nasal inhalation, such as in the form of purified vapors or by smoking of Cannabis.

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

Pharmaceutical formulations adapted for rectal administration may be presented as suppositories or enemas.

Pharmaceutical formulations adapted for vaginal administration may be presented as pessaries, tampons, creams, gels, pastes, foams or spray formulations.

According to certain embodiments, pharmaceutical compositions comprising Cannabidiol, or any derivative thereof according to the present invention are also useful for parenteral administration, i.e., subcutaneously (s.c.), intramuscularly (i.m.), and intravenously (i.v.). The compositions for parenteral administration commonly comprise a solution of Cannabidiol dissolved in an acceptable carrier.

In one embodiment, the compositions of the invention are suitable for oral administration. The Cannabidiol compositions can be administered from one or more times per day to one or more times per week, including once every other day. Dosing is dependent on the severity of the symptoms and on the responsiveness of the subject to the treatment. The skilled artisan will appreciate that certain factors may influence the dosage and timing required to effectively treat a subject, including but not limited to the severity of the disease, previous treatments, the general health and/or age of the subject, and other diseases present.

The present invention relates to the treatment of subjects, or patients, in need thereof. By “patient” or “subject in need” it is meant any mammal for which administration of the composition of the invention is desired, in order to prevent, overcome or slow down a medical condition. As described herein, the medical condition for treatment includes Grade III or Grade IV acute GVHD, severe chronic GVHD, refractory forms of either acute or GVHD, or any of the conditions described herein that are associated with acute or GVHD (e.g. pathologies of the skin, liver, lungs, and the like).

The terms “treatment”, “prevention” and “prophylaxis” refer to the complete range of therapeutically positive effects of administrating to a subject including inhibition, reduction of, alleviation of, and relief from, GVHD, specifically, acute and chronic GVHD. More specifically, treatment or prevention includes the prevention or postponement of development of the disease, prevention or postponement of development of symptoms and/or a reduction in the severity of such symptoms that will or are expected to develop.

These further include ameliorating existing symptoms, preventing additional symptoms and ameliorating or preventing the underlying causes of symptoms.

It should be noted that particularly in case of human subjects, administration of the compositions of the invention to the patient includes both self-administration and administration to the patient by another person.

To provide a “preventive treatment” or “prophylactic treatment” is acting in a protective manner, to defend against or prevent something, especially a condition or disease.

As used herein, “disease”, “disorder”, “condition” and the like, as they relate to a subject's health, are used interchangeably and have meanings ascribed to each and all of such terms.

The term “pharmaceutical composition” refers to an active compound in any form suitable for effective administration to a subject, e.g., a mixture of the compound and at least one pharmaceutically acceptable carrier.

The term “therapeutically effective amount” is intended to mean that amount of a drug or pharmaceutical agent that will elicit the biological or medical response of a tissue or human that is being sought by a researcher, medical doctor, or other clinician, or by the subject himself.

As used herein, a “pharmaceutically acceptable carrier” means a carrier or diluent that does not cause significant irritation to a subject and does not abrogate the biological activity and properties of the administered compound.

A “pharmaceutically acceptable excipient” means an inert substance added to a pharmaceutical composition to further facilitate administration of the compound. Examples, without limitation, of excipients include calcium carbonate, calcium phosphate, various sugars and types of starch, cellulose derivatives, gelatin, vegetable oils and polyethylene glycols.

The following examples, which further describe the invention, are offered by way of illustration and are not intended to limit the invention in any manner.

### EXAMPLES

### Example 1: Prevention of Acute and Chronic GVHD in Allogeneic Stem Cell Transplanted Patients

Forty six (46) patients over 18 years of age undergoing allogeneic hematopoietic stem cell transplantation at the Rabin Medical Center in Israel were recruited. Patients having history of psychosis or asthma, or consuming Cannabis during the last two months before transplantation were excluded from the study.

All patients received standard GVHD prophylaxis consisting of cyclosporine twice a day starting on day −1 with target trough levels ≥200 ng/mL in combination with short course of methotrexate (15 mg/sqm on day +1 and 10 mg/sqm on days +3 and +6). Most patients transplanted from unrelated donors received anti-T-cell globulin (ATG Fresenius) at a low dose of 5 mg/kg on days −3 to −1.

The investigational agent, CBD (STI Pharmaceuticals, Brentwood, Essex, UK) was dissolved in olive oil at a concentration of 2.5% and was orally administered from day −7 through day +30. The starting dose of CBD was 10 mg twice a day, and doses were doubled every other day to a maximal dose of 150 mg twice a day.

To estimate the prophylactic and therapeutic effect of Cannabidiol, the patients were carefully monitored and documented for the presentation of acute and chronic GVHD symptoms, the time of onset, the severity of the symptoms, the responsiveness to treatment, and the occurrence of infections.

Furthermore, a panel of 4 serum cytokines (soluble TNF receptor 1 (sTNFRI), soluble IL-2 receptor alpha (sIL2R-alpha), hepatocyte growth factor (HGF), and IL8)) were monitored. Blood samples were taken at days −7(A), 0(B), +14(C), and +28(D). The difference in blood levels between the various time points (B-A, C-B and D-C) were assessed.

Primary end points were safety and cumulative incidence of grade 2-4 and grade 3-4 GVHD by day +100. The secondary end points were cumulative incidence of chronic and severe chronic GVHD, non-relapse mortality (NRM), relapse incidence (RI), and overall survival (OS). NRM mortality was defined as death while the patient was in CR. Cumulative incidence curves were used for acute GVHD and chronic GVHD estimation, taking into account relapse and death as competing risks. Cumulative incidence curves were used for RI and NRM taking into account disease recurrence and death as competing events. Probabilities of OS and LFS were calculated using the Kaplan-Meier estimates.

Median age was 56 (range, 22 to 73) years. Most patients (n=38, 79%) had acute leukemia and 13 patients (27%) had chemo-refractory disease at transplantation. Twenty patients (42%) received allografts from unrelated donors. Thirty-five patients (73%) received myeloablative conditioning. Median follow-up of survivors was 10 (range, 1.3 to 18) months. Drug compliance was good, with 32 patients (70%) taking 100% of the doses. Noncompliance with study medication was mainly due to mucositis and nausea. Median dose dense among non-compliant patients was 86% (range, 43%-96%). No grade 3 to 4 toxicities related to CBD were observed.

Six patients developed grade 2 to 4 acute GVHD. Two patients had a gut-only involvement, 3 patients had involvement of skin and gut, and 1 patient had involvement of gut and liver. Median time to onset of grade 2 to 4 acute GVHD was 60 days (range, 41 to 150 days). Cumulative incidence rates of grade 2 to 4 acute GVHD at day 100 was 12.4%. Cumulative incidence rates of grade 3 to 4 acute GVHD at day 100 was 5%.

Among patients surviving more than 100 days after HCT (n=33), chronic GVHD occurred in 11 patients (overlap, n=3 and classic, n=8), with a median time to onset of 159 (range, 125 to 335) days. Of the 11 patients, 8 had limited chronic GVHD with a low stage involvement of the oral mucosa and a mild elevation of liver enzymes. In the 3 patients with severe chronic GVHD, organ involvement included eyes (n=3), mouth (n=3), skin (n=3), lungs (n=2) and musculoskeletal (n=1). Cumulative incidence rates of overall and extensive chronic GVHD at 1 year were 43% and 19%, respectively.

Cumulative incidences of relapse at 100 days, 6 months and 12 months after HCT were 12%, 22%, and 31%, respectively. Non-relapse mortality at 100 days, 6 months and 12 months after HCT were 8.1%, 11.6%, and 11.6%, respectively. Overall survival rates at 100 days, 6 months and 12 months after HCT were 84%, 76% and 70%, respectively.

Interestingly, patients with increased D-C serum levels of IL8 and sIL2R-alpha had a relative risk of 3.8 (95% CI 0.8-17.1, p=0.1) and 2.8 (95% CI 1.1-7.5, p=0.05), respectively, for developing chronic GVHD.

The results obtained clearly indicate that administration of Cannabidiol reduces the incidence and severity of acute and chronic GVHD after allogeneic stem cell transplantation. The findings presented herein demonstrate the advantages concurrent with low toxicity and lack of psychotropic effects of Cannabidiol in preventing acute and chronic GVHD.

### Example 2: CBD Treatment for Chronic GVHD

Twelve patients over 18 years of age with extensive chronic GVHD after allogeneic HCT were recruited at the Rabin Medical Center in Israel. Patients having a history of psychosis or asthma, or consuming Cannabis during the last two months before transplantation were excluded from the study.

Patients were presently treated with a calcineurine inhibitor (cyclosporine or tacrolimus) with or without oral prednisone.

Patients were given oral CBD 50 mg twice a day.

A clinical improvement was documented in 6 out of 8 patients with skin involvement, 2 out of 3 with lung involvement, 3 out 4 with liver involvement and 3 out of 3 with oral involvement.

The results obtained clearly indicate that administration of CBD reduces the severity of chronic GVHD.

### Example 3: Cannabidiol (CBD) for Treatment of Severe (Grade 3-4) and/or Refractory Acute GVHD

This example describes cases studies that show the surprising efficacy of CBD treatment of patients experiencing severe acute GVHD.

A 45 year old male with acute myeloid leukemia in second complete remission, underwent an allogeneic hematopoietic cell transplantation from a matched sibling female donor with a myeloablative conditioning consisting of fludarabine (160 mg/m²) and busulfex (12.8 mg/kg). One month post-transplantation he developed classic grade 4 acute GVHD of the gastro-intestinal tract (GIT) confirmed by intestinal biopsy. He was refractory to cyclosporine and high-dose steroids, and was then given second line therapy consisting of mycophenolate mofetil followed by extra-corporeal photopheresis (ECP), from which a partial response was obtained. The patient then developed severe myopathy and became bed restricted. During tapering off of steroids he had relapse of grade 4 acute GVHD with GIT and skin involvement. He was refractory to high-dose steroids and thus started oral cannabidiol (CBD) 150 mg twice a day. After 14 days of CBD he had complete cessation of diarrhea and resolution of his skin rash. CBD was stopped several weeks later upon remission. Eighteen months following cessation of treatment, the patient has no symptoms or signs of acute GVHD. Nevertheless, he developed chronic GVHD for which he was given low dose cyclosporine and low dose prednisone.

A 31-year-old male with Philadelphia positive acute lymphoid leukemia in first complete remission underwent an allogeneic hematopoietic cell transplantation from a male matched unrelated donor with reduced intensity conditioning consisting of fludarabine (150 mg/m²), busulfex (12.8 mg/kg) and ATG (Fresenius, 15 mg/kg). Two months after transplantation, he developed generalized erythroderma ranked as stage 3 and grade 2 acute GVHD of the skin, which was confirmed by skin biopsy. Due to refractoriness to tacrolimus, systemic and topical steroids he started ECP with some improvement but without remission. He was still on tacrolimus and steroids when 11 months post-transplantation he had severe worsening of his skin rash with generalized erythroderma and desquamation ranked as stage 4 and grade 4 acute GVHD of the skin. He started oral CBD 150 mg twice a day and prednisone 40 mg once a day. Skin rash and desquamation improved promptly until complete resolution. CBD was stopped 2 months later after obtaining complete remission of acute GVHD. Tacrolimus and steroids were gradually tapered off until complete cessation.

A 39 year old female with acute myeloid leukemia in first complete remission, underwent an allogeneic hematopoietic cell transplantation from a male matched sibling donor with myeloablative conditioning consisting of busulfex (12.8 mg/kg) and cyclophosphamide (120 mg/kg). Eight months post-transplantation, leukemia relapsed. She received salvage chemotherapy followed by donor lymphocyte infusion (DLI) and achieved a complete hematologic remission. One month post DLI she developed jaundice with bilirubin levels of 11.5 mg % (normal ≤1 mg %) and biopsy proven late onset acute GVHD of the liver ranked as stage 3 and grade 3. She was refractory to high-dose steroids and started oral CBD 150 mg twice a day. Jaundice resolved within few days and bilirubin dropped to normal levels (0.7-1.3 mg %). Steroids could be tapered off until complete cessation Thus reducing the therapeutically effective dose of a steroid can be a process wherein the steroid dose administered with CBD is gradually decreased over time.

The above case studies demonstrate not only that CBD treatment is effective for treatment of acute Grade 3 and Grade 4 GVHD, but that it provides complete treatment of the most severe forms of GVHD for which a cure is otherwise not possible. The present CBD treatment, unexpectedly, rendered a refractory dose of a steroid-therapeutically effective. Moreover, this example demonstrates that CBD is effective in treatment of severe acute GVHD as it occurs in all effective organs.

### Example 4: Cannabidiol (CBD) for Treatment of Severe Refractory Chronic GVHD

This example describes cases studies that show the surprising efficacy of CBD treatment of patients experiencing severe refractory chronic GVHD.

A 35-year-old male with acute lymphoid leukemia in first complete remission, underwent an allogeneic hematopoietic cell transplantation from a female matched unrelated donor with myeloablative conditioning consisting of total body irradiation (12 Gray), cyclophosphamide and ATG (Fresenius, 15 mg/kg). The patient developed an overlap syndrome (concomitant acute plus chronic GVHD features) during the first month after transplantation which was treated with cyclosporine and steroids until complete remission. Immunosuppression was stopped 5 months after transplantation. Eighteen months after transplantation he developed chronic extensive GVHD with involvement of the oral cavity, eyes, liver, joints and skin. Skin involvement progressed to sclerosis. He started again cyclosporine and oral steroids with no improvement. There was no further improvement with imatinib (Gleevec). He started oral CBD, as prepared above, at 75 mg twice a day, and after several weeks of treatment he resolved his liver and oral GVHD and significantly improved skin sclerosis and joint range of motion. This outcome, unexpectedly and clearly demonstrated that CBD was absolutely necessary to render the refractory steroid therapy—effective. Unexpectedly, Prednisone effective therapeutic dose was tapered off up to 7.5-10 mg QD (20-50% reduction in the steroid's dose).

A 35-year-old female with acute myeloid leukemia in second complete remission underwent an allogeneic hematopoietic cell transplantation from a matched female sibling donor with myeloablative conditioning consisting of busulfex (12.8 mg/kg) and cyclophosphamide (120 mg/kg). The patient experienced relapse in the central nervous system and breasts 12 months after transplantation, received chemo-radiotherapy and achieved complete remission. Shortly after remission, she developed chronic extensive GVHD with severe involvement of the eyes, joint and skin. Skin involvement progressed to severe sclerosis with limited range of motion of the joints. She was unresponsive to cyclosporine and oral steroids and therefore they were stopped after few months. She started oral CBD 75 mg BID and after several months she had significant improvement of her skin and joints range of motion. Interestingly and unexpectedly, these clinical outcomes show that CBD has a significant therapeutic effect in treating GVHD with and without steroid treatment.

### Example 5: Cannabidiol (CBD) Reduced the Actual Effective Dose of Steroids and Renders Ineffective Dose, Therapeutically Effective

63-year-old female with secondary acute myeloid leukemia in 1^{st} complete remission, underwent and allogeneic hematopoietic cell transplantation from a male matched unrelated donor with a myeloablative conditioning consisting of fludarabine (160 mg/m²), busulfex (12.8 mg/kg) and ATG (Fresenius, 15 mg/kg). Eleven months after transplantation she developed jaundice with bilirubin level of 4.2 mg % and biopsy proven late onset acute GVHD of the liver ranked as stage 2 and grade 3. She started tacrolimus and high-dose steroids and had a partial response for a short period of time. Two months later (13 months post transplantation), while still given tacrolimus and steroids she had an upsurge in bilirubin to 8.5 mg %, ranked as stage 3 and grade 3 acute GVHD of the liver. She started oral CBD 150 mg BID. Jaundice resolved gradually and bilirubin dropped to normal levels. Steroids could be tapered off to a low dose of 7.5 mg QD.

A 58-year-old male with refractory follicular lymphoma underwent an allogeneic hematopoietic cell transplantation from a male matched unrelated donor with a reduced intensity conditioning consisting of fludarabine (150 mg/m²), melphalan (100 mg/m²) and ATG (Fresenius 15 mg/kg) and achieved a complete remission. Two years later he relapsed and received salvage immune-chemotherapy with bendamustine and rituximab followed by DLI and achieved a complete remission. One month later he was admitted with severe late onset acute GVHD with skin (rash) and upper (nausea and vomiting) plus lower gastrointestinal involvement (bloody diarrhea, crampy abdominal pain and severely inflamed and ulcerated mucosa). Biopsy proven GVHD was ranked as stage 3 of the skin and stage 4 and grade 4 acute GVHD of the GIT. He started cyclosporine and high-dose steroids. Despite skin response, GVHD of the GIT was refractory to prolonged systemic treatment of steroids. He started oral CBD 150 mg BID, symptoms improved gradually, and a very good partial response (VGPR) was attained to the steroids. Prednisone was tapered off to 20 mg QD (40%)—found to be therapeutically effective. Two months later diarrhea resumed following discontinuing CBD without doctors' approval. A second VGPR was obtained upon renewal of methylprednisolone 0.5 mg/kg QD and CBD 150 mg BID.

### Example 6: Cannabidiol (CBD) for Treating Severe and Critical ARDS (COVID-19 Infection)

An open label phase 1/2 study was performed for evaluating Cannabidiol and steroid combined treatment for severe and critical COVID-19 pulmonary infection.

Current management of COVID-19 is mainly supportive, and respiratory failure from acute respiratory distress syndrome (ARDS) is the leading cause of mortality. Unfortunately, there is currently no effective antiviral therapy for COVID-19 and treatment is merely supportive. Cytokines and chemokines are thought to play an important role in immunity and immunopathology during virus infections. Patients with severe COVID-19 have higher serum levels of pro-inflammatory cytokines (TNF-α, IL-1 and IL-6) and chemokines (IL-8) compared to individuals with mild disease or healthy controls, similar to patients with SARS. The change of laboratory parameters, including elevated serum cytokine and chemokine levels in infected patients are correlated with the severity of the disease and adverse outcome, suggesting a possible role for hyper-inflammatory responses in COVID-19 pathogenesis. Thus, the virus may be regarded as a provocateur, which triggers a disproportionate and uncontrolled immune response, leading to potentially life-threatening damage to lung tissue.

Methods and Patients

Patient population included PCR confirmed covid-19 patients. Eleven (11) patients with severe and critical COVID-19 infection were recruited and treated with standard of care (including prednisone) and CBD at a dose of 150 mg BID.

Results:

TABLE 4

| | |
|---|---|
| Control (SOC + | CBD + SOC + |
| steroids) | with steroids |

| | N | % | N | % |
|---|---|---|---|---|
| Additional | 12 | 54.5 | 4 | 36.3 |
| positive | | | | |
| microbiology | | | | |
| result | | | | |
| Length of | 22 | Median | 11 | Median |
| hospitalization | | 23 days | | 14 days |
| Mortality | 13 | 59.1 | 3 | 27.3 |

### N: Number of Patients

Based on a matched pair analysis (1:2 CBD vs control) it was shown that the death rate observed for the CBD group (27%) was significantly lower than the rate observed for the control group (59%). Regarding the length of stay in hospital (LOS-H), a significant shorter stay was observed in the CBD group—a mean of 15 days compared to 37 days in the control group. Thus, the CBD treated group recovered much faster.

The change from baseline of the last observation in Systolic BP, Lym (%) and FiO2% was found statistically different between treatment groups.

TABLE 5

| | |
|---|---|
| Control | CBD |
| (including | (including |
| steroid) | steroid) |

| | N | % | N | % |
|---|---|---|---|---|
| Respiratory support | | | | |
| Supplemental oxygen | 22 | 100 | 11 | 100 |
| HFNC | 20 | 90.9 | 11 | 100 |
| Invasive mechanical | 16 | 72.7 | 4 | 36.3 |
| ventilation | | | | |
| Medical treatment | | | | |
| Dexamethasone | 22 | 100 | 11 | 100 |
| Remdesivir | 9 | 40.9 | 8 | 72.7 |
| Actemra | 3 | 13.6 | 1 | 9 |
| Antibiotic treatment | 17 | 77.2 | 5 | 45.4 |
| Vasopressors | 16 | 72.7 | 4 | 36.3 |
| Additional support | | | | |
| ECMO | 4 | 18.2 | 0 | 0 |
| Renal replacement | 1 | 4.5 | 3 | 27.3 |
| therapy | | | | |

### Example 7: Cannabidiol (CBD) for Reducing Steroid Dose in Treating and Controlling Steroid-Dependent Crohn's Disease (CD)

Crohn's Disease is a relapsing inflammatory disease, mainly affecting the gastrointestinal tract, and frequently presents with abdominal pain, fever, and clinical signs of bowel obstruction or diarrhea with passage of blood or mucus or both. The diagnosis of CD is based on a combination of clinical symptoms, laboratory tests and imaging data. CD seems to result from an impaired interaction of the intestinal commensal microbiota that is normally in a state of symbiotic mutualism with the human host.

Methods and Patients:

Patient population included patients with biopsy confirmed, active, steroid dependent Crohn's Disease, currently on a stable dose of steroids for at least 2 weeks.

The study was performed in 3 stages: 1. a run-in stage where individual patient Standard of Care (steroid therapy with or without additional therapies) was administered in a stable fashion during the first weeks of the study in addition to increasing doses of CBD or placebo in a double blind (DB) manner. Target dose of CBD was 300 mg.

2. patients continued receiving this 300 mg dose/placebo for an additional period of 3 months. Weekly to 3-days tapering off steroids was commenced during this period.

3. An interim analysis per patient was performed in order to decide if the patient should continue, stop or crossover from placebo to CBD arm. Patients continued treatment until 12 months from study initiation.

Results:

The analysis of the primary endpoint included an intention to treat (ITT) analysis in 13 patients, 8 were in the CBD group (CBD and steroids) and 5 in the placebo group (steroid and placebo). A per protocol (PP) analysis in 11 patients was also performed, 7 in the CBD group and 4 in the placebo group.

TABLE 6

| | Placebo (steroid | | CBD (with | | | |
|---|---|---|---|---|---|---|
| | and placebo) | | steroid) | | All | |
| | N | % | N | % | N | % |
| Intend-to-Treat (ITT) | | | | | | |
| Yes | 5 | 100.0 | 8 | 100.0 | 13 | 100.0 |
| Per Protocol (PP) | | | | | | |
| No | 1 | 20.0 | 1 | 12.5 | 2 | 15.4 |
| Yes | 4 | 80.0 | 7 | 87.5 | 11 | 84.6 |

In the per protocol analysis, two patients were excluded from the analysis. Exclusion was performed before un-blinding of the randomization list.

Intention to Treat (ITT) Analysis

In an intention to treat (ITT) analysis in 13 patients, 62.5% of patients treated with CBD (and steroid) reached the primary endpoint as compared to 20% in the placebo group (steroid and placebo). The primary endpoint of the study was defined as 50% reduction in dose of steroids without flares and without addition of Crohn's Disease specific drugs without an increase of more than 70 points in the CDAI scores at week 15.

TABLE 7

| Primary endpoint: 50% reduction in dose of | | | | |
|---|---|---|---|---|
| steroids without flares and without addition | | | | |
| of CD specific drugs without an increase of | | | | |
| more than 70 points in the CDAI scores at | Placebo | | CBD | |
| week 15 | N | % | N | % |
| No | 4 | 80.0 | 3 | 37.5 |
| Yes | 1 | 20.0 | 5 | 62.5 |

### Per Protocol Analysis:

In the PP analysis in 11 patients that completed at least 15 weeks of treatment or dropped out due to flares or AEs, 71.4% of patients in the CBD group reached the primary endpoint as compared to 25% in the placebo group.

TABLE 8

| 50% reduction in dose of steroids without | | | | |
|---|---|---|---|---|
| flares and without addition of CD specific | | | | |
| drugs without an increase of more than 70 | Placebo | | CBD | |
| points in the CDAI scores at week 15 | N | % | N | % |
| No | 3 | 75.0 | 2 | 28.6 |
| Yes | 1 | 25.0 | 5 | 71.4 |

In the per protocol analysis, ten out of ten patients (100%) in the CBD group were tapered off completely from steroid treatment, as compared to 1 out of 4 (25%) in the placebo group.

Adverse events (AEs): In the CBD group most patients did not have any AEs. One patient had elevated liver enzymes and diarrhea. Another patient had elevated glucose levels. In the placebo group, one patient had rectal bleeding and stomach pain. Another patient had agitation, memory loss and chest pain.

Summary

62.5% of patients in the CBD group reached the primary endpoint as compared to 20% in the placebo group. In a PP analysis 71.4% of patients in the CBD group (treated with CBD and steroid but at sub-optimal or reduced dose) reached the primary endpoint as compared to 25% in the placebo group (placebo and steroid without CBD). Additionally, 100% of patients in the CBD group were tapered off steroids completely as compared to 25% in the placebo group. 75% of patients in the placebo had flares and either crossed over to the CBD group or dropped out of the study.

### Example 8: Cannabidiol (CBD) for Reducing Steroid Dose in Treating Steroid-Dependent Urticaria

A clinical study was performed in 15 patients. A preliminary per protocol analysis was performed in 9 patients that completed at least 10 weeks of treatment.

In the study safety, tolerability and trends of efficacy of the CBD treatment were evaluated, as measured by the total steroid courses needed during CBD administration period per patient versus the total steroid courses 6 months before treatment as well as the intensity of the disease measured by the Urticaria Activity Score (UAS7).

The Disease:

Chronic spontaneous urticaria (CSU) is a mast cell-driven disease that is defined as the occurrence of wheals, angioedema, or both for more than 6 weeks. CSU affects up to 1% of the general population, exerting a devastating impact on patients' quality of life. Histamine and other mediators, such as platelet-activating factor (PAF) and cytokines released from activated skin mast cells, result in sensory nerve activation, vasodilatation and plasma extravasation as well as cell recruitment to urticarial lesions. Histologically, wheals are characterized by edema of the upper and mid dermis, with dilatation and augmented permeability of the postcapillary venules, as well as lymphatic vessels of the upper dermis leading to leakage of serum into the tissue.

Methods and Patients:

15 patients with active CSU for at least 4 months were recruited. Only patients that had been which was treated for the last 4 months with antihistamines as well as at least two courses of steroids (ex. Prednisone) were recruited.

Study Design: Each patient commenced the study with a one month run-in period in which he/she was administered individual patient Standard of Care (SOC): anti-histamines and steroids as needed (PRN, pro re nata) plus placebo (olive oil). At the end of the one-month run-in period, subjects continued individual SOC plus increasing doses of CBD during the first six weeks of the study up to 300 mg.

At the conclusion of the CBD dose escalation segment of the study, patients continued receiving 300 mg CBD+Anti-histamines PRN for an additional follow-up period of three months. In the case of flare, patients were treated with higher dose of Antihistamines and steroids if needed in addition to CBD. Each patient served as his/her own control.

Preliminary Results:

All patients enrolled in the study were administered at least 2 courses of steroids 6 months prior to the CBD treatment. During the CBD treatment, steroid dose was continuously reduced until steroid treatment was completely tapered off or were not administered at all in ALL patients analyzed.

TABLE 9

| | Combined steroid and CBD treatment |
|---|---|
| patient 1 | tapered off completely |
| patient 2 | tapered off completely |
| patient 3 | tapered off completely |
| patient 5 | tapered off completely |
| patient 8 | tapered off completely |
| patient 9 | tapered off completely |
| patient 10 | tapered off completely |
| patient 11 | tapered off completely |
| patient 14 | tapered off completely |

In the PP analysis in 9 patients that completed at least 10 weeks of treatment (visit 6), a reduction in the UAS7 was shown as depicted by the relative change (%) in UAS7 from Visit 1 (week 1—start of run in period) and Visit 2 (week 4—start of CBD treatment with steroid).

| | | Relative |
|---|---|---|
| | Relative | change from |
| | change from | Visit 2 - Week |
| | Visit 1 - Week | 4 (start CBD |
| | 1 (start run-in) | with steroid) |
| | [%] | admin) [%] |
| Visit 1 Week 1 (start run-in) | 0 | 0 |
| Visit 2 Week 4 (start CBD) | 0.67 | 8.82 |
| Visit 3 Week 6 | 44.33 | 29.57 |
| Visit 4 Week 8 | 49.68 | 2.43 |
| Visit 5 Week 10 | 46.32 | 38.87 |
| Visit 6 Week 12 | 64.29 | 53.54 |
| Visit 7 Week 14 | 78.57 | 60.31 |
| Visit 8 Week 16 | 35.71 | 27.68 |
| Visit 9 Week 18 | 71.43 | 21.72 |
| Visit 10 Week 20 | 85.71 | 49.76 |
| Visit 11 Week 22 | 100 | 74.6 |
| Visit 12 Week 24 | 100 | 17.86 |

### IN CONCLUSION

In this study, in 9 patients receiving CBD after being administered various steroids courses to control flares, the disease was controlled and surprisingly, steroid treatment was gradually tapered off (CBD and steroid combined treatment as described herein) until it was completely tapered off (steroid not administered at all in ALL patients analyzed), nonetheless all patients tapered off (now treated with CBD with no steroid) exhibited clinical outcomes as if they are treated with an effective steroid dose.

Interestingly, a reduction in the Urticaria score from Visit 1 (week 1—start of run-in period) and Visit 2 (week 4—start of CBD treatment) was recorded.

What is claimed is:

1. A method for enhancing the therapeutic effect of a steroid in a subject afflicted with acute respiratory distress syndrome (ARDS), inflammatory bowel disease (IBD), or Urticaria, comprising administering to said subject said steroid and cannabidiol (CBD), thereby enhancing the therapeutic effect of a steroid in a subject afflicted with acute respiratory distress syndrome (ARDS), inflammatory bowel disease (IBD), or Urticaria.

2. The method of claim 1, wherein said enhancing the therapeutic effect of said steroid comprises: reducing a side effect associated with said steroid or rendering a refractory steroid dose a therapeutically effective dose.

3. The method of claim 1, wherein said enhancing the therapeutic effect of said steroid is reducing the effective dose of said steroid.

4. The method of claim 1, wherein said steroid, said CBD, or any combination thereof is administered systemically.

5. The method of claim 1, wherein said steroid, said CBD, or any combination thereof is administered orally or topically.

6. The method of claim 1, wherein the CBD is administered daily in doses of between 5 mg to 600 mg.

7. The method of claim 1, wherein said enhancing the therapeutic effect of said steroid is enhancing the recovery of a subject afflicted with said ARDS or reducing death risk of said subject afflicted with said ARDS.

8. The method of claim 1, wherein said ARDS is COVID-19.

9. The method of claim 7, wherein said subject is treated with respiratory support, ventilatory support or a combination thereof.

10. A method of tapering off steroid therapy in a subject afflicted with a chronic inflammatory disease, comprising the following consecutive steps: (a) administering to said subject CBD and an effective dose of said steroid; (b) gradually decreasing the dose of said steroid and maintaining co-administration of said CBD, wherein the rate of said decreasing is matched to controlling said chronic inflammatory disease; and (c) tapering off said steroid therapy while administering said CBD, once said chronic inflammatory disease is controlled, inhibited, treated or ameliorated.

11. The method of claim 10, wherein said chronic inflammatory disease is an inflammatory bowel disease (IBD) or Urticaria.

12. The method of claim 11, wherein said IBD is Crohn's disease.

13. The method of claim 10, wherein said inhibited comprises inhibiting flare ups of said inflammatory disease.

14. The method of claim 10, wherein said tapering off said steroid therapy is ceasing said steroid therapy.

15. The method of claim 10, wherein said chronic inflammatory disease in said subject prior to said tapering, is controlled, inhibited, treated or ameliorated by said effective dose of said steroid.

16. The method of claim 10, wherein said chronic inflammatory disease in said subject prior to said tapering, is controlled, inhibited, treated, or ameliorated by said effective dose of said steroid in the absence of said CBD.

17. The method of claim 10, wherein CBD dose in step (b) is increased concomitantly with decreasing said dose of said steroid.

18. The method of claim 10, wherein said tapering off steroid therapy is further reducing daily dose of an antihistamine, wherein step (a) comprises administering to said subject CBD, an effective dose of antihistamine, and an effective dose of said steroid; wherein step (b) comprises gradually decreasing the dose of said steroid and said antihistamine and maintaining co-administration of said CBD, wherein the rate of said decreasing is matched to controlling said chronic inflammatory disease; wherein step (c) comprises tapering off said steroid therapy and said reducing daily dose of said antihistamine while administering said CBD, once said chronic inflammatory disease is controlled, inhibited, treated or ameliorated.

19. The method of claim 18, wherein said chronic inflammatory disease comprises chronic Urticaria.
